(19) United States
(12) Patent Application Publication
(10) Pub. No.: US 2020/0323849 A1
(43) Pub. Date: Oct. 15, 2020

(54) COMBINATIONS OF HISTONE DEACETYLASE INHIBITORS AND IMMUNOMODULATORY DRUGS

(71) Applicants: Acetylon Pharmaceuticals, Inc., Boston, MA (US); DANA-FARBER CANCER INSTITUTE, Boston, MA (US)

(72) Inventors: Steven Norman Quayle, Brookline, MA (US); Simon Stewart Jones, Harvard, MA (US); Kenneth C. Anderson, Wellesley, MA (US); Teru Hideshima, Brookline, MA (US)

(21) Appl. No.: 16/684,809

(22) Filed: Nov. 15, 2019

Related U.S. Application Data
(63) Continuation of application No. 14/508,072, filed on Oct. 7, 2014, now abandoned.
(60) Provisional application No. 61/911,089, filed on Dec. 3, 2013.
(60) Provisional application No. 61/889,640, filed on Oct. 11, 2013.

(51) Int. Cl. A61K 31/505; A61K 31/454; A61K 31/573; C07D 239/42; C07D 401/04

(52) U.S. Cl. CPC A61K 31/505; A61K 31/454; C07D 401/04; C07D 239/42; A61K 31/573

(57) ABSTRACT

The invention relates to combinations comprising an HDAC inhibitor and an immunomodulatory drug for the treatment of multiple myeloma in a subject in need thereof. The combinations may, optionally, further comprise an anti-inflammatory agent, such as dexamethasone. Also provided herein are methods for treating multiple myeloma in a subject in need thereof comprising administering to the subject an effective amount of one of the above combinations.

### CROSS-REFERENCE TO RELATED APPLICATIONS

This application is a continuation of U.S. application Ser. No. 14/508,072, filed Oct. 7, 2014, which application claims priority to U.S. Provisional Application Ser. No. 61/889,640, filed Oct. 11, 2013, and U.S. Provisional Application Ser. No. 61/911,089, filed Dec. 3, 2013, each of which is incorporated herein by reference in its entirety.

### BACKGROUND

Histone deacetylase (HDAC) enzymes represent attractive therapeutic targets in multiple myeloma, but unfortunately non-selective HDAC inhibitors have led to dose-limiting toxicities in patients.

The immunomodulatory (IMiD) class of drugs, including lenalidomide and pomalidomide, exhibit striking anti-myeloma properties in a variety of multiple myeloma models, and have demonstrated significant clinical activity in multiple myeloma patients.

Prior studies have shown clinical activity of a combination of the non-selective HDAC inhibitor vorinostat with lenalidomide and dexamethasone in myeloma patients (Richter, et al., ASH, 2011). However, many patients experienced significant toxicities with this regimen that significantly limits its clinical utility.

Due to the dose-limiting toxicities of the above therapies, there is an ongoing need in the art for more efficacious and less toxic compositions and methods for the treatment of multiple myeloma. In order to meet these needs, provided herein are pharmaceutical combinations comprising a HDAC inhibitor and an immunomodulatory drug, and methods for the treatment of multiple myeloma. The combinations and methods of the invention are well tolerated and do not exhibit the dose-limiting toxicities of prior therapies.

### SUMMARY OF THE INVENTION

Provided herein are pharmaceutical combinations for the treatment of multiple myeloma in a subject in need thereof. Also provided herein are methods for treating multiple myeloma in a subject in need thereof.

Provided in some embodiments are combinations comprising a histone deacetylase (HDAC) inhibitor and an immunomodulatory drug (IMiD) for the treatment of multiple myeloma in a subject in need thereof. In some specific embodiments, the combinations do not include dexamethasone. In other specific embodiments, the combinations further comprise an anti-inflammatory agent, such as dexamethasone.

For example, an embodiment of the invention provides a pharmaceutical combination for treating multiple myeloma comprising a therapeutically effective amount of a histone deacetylase 6 (HDAC6) specific inhibitor or a pharmaceutically acceptable salt thereof, and an immunomodulatory drug (IMiD) or a pharmaceutically acceptable salt thereof, wherein the combination does not include dexamethasone.

Provided in other embodiments are methods for treating multiple myeloma in a subject in need thereof comprising administering to the subject an effective amount of a combination comprising a histone deacetylase (HDAC) inhibitor and an immunomodulatory drug (IMiD). In some specific embodiments of the methods, the combinations do not include dexamethasone. In other specific embodiments of the methods, the combinations further comprise an anti-inflammatory agent, such as dexamethasone.

For example, an embodiment of the invention provides a method for treating multiple myeloma in a subject in need thereof comprising administering to the subject a therapeutically effective amount of a pharmaceutical combination comprising a histone deacetylase 6 (HDAC6) specific inhibitor or a pharmaceutically acceptable salt thereof, and an immunomodulatory drug (IMiD) or a pharmaceutically acceptable salt thereof, wherein the combination does not include dexamethasone.

In specific embodiments, the HDAC6 specific inhibitor is a compound of Formula I:

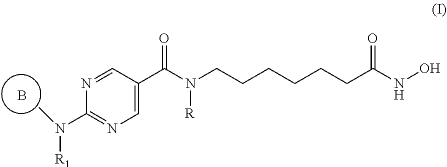

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl.

In preferred embodiments, the compound of Formula I is:

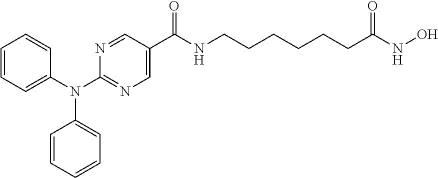 [SMILES: O=C(CCCCCCNC(=O)c1cnc(N(c2ccccc2)c2ccccc2)nc1)NO]

or a pharmaceutically acceptable salt thereof.

In yet other embodiments, the compound of Formula I is:

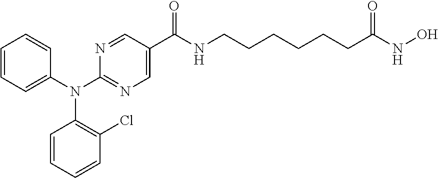 [SMILES: O=C(CCCCCCNC(=O)c1cnc(N(c2ccccc2)c2ccccc2Cl)nc1)NO]

or a pharmaceutically acceptable salt thereof.

In other specific embodiments, the HDAC6 specific inhibitor is a compound of Formula II:

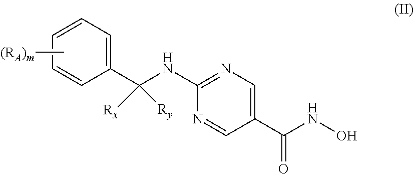

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂;

and
m is 0, 1, or 2.

In preferred embodiments, the compound of Formula II is:

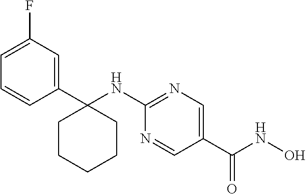 [SMILES: O=C(NO)c1cnc(NC2(c3cccc(F)c3)CCCCC2)nc1]

or a pharmaceutically acceptable salt thereof.

In other preferred embodiments, the compound of Formula II is:

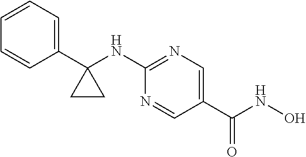 [SMILES: O=C(NO)c1cnc(NC2(c3ccccc3)CC2)nc1]

or a pharmaceutically acceptable salt thereof.

In some embodiments of the combinations and/or methods, the immunomodulatory drug is a compound of Formula III:

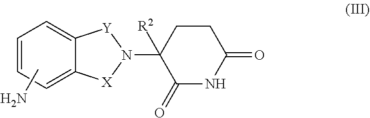

or a pharmaceutically acceptable salt thereof,
wherein,
one of X and Y is C═O, the other of X and Y is CH₂ or C═O; and
R² is H or C₁₋₆-alkyl.

In preferred embodiments, the compound of Formula III is:

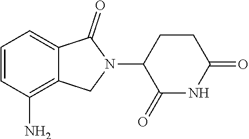 [SMILES: Nc1cccc2c1CN(C1CCC(=O)NC1=O)C2=O]

or a pharmaceutically acceptable salt thereof.

In yet other preferred embodiments, the compound of Formula III is:

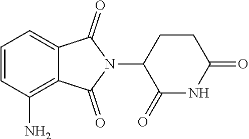 [SMILES: Nc1cccc2c1C(=O)N(C1CCC(=O)NC1=O)C2=O]

or a pharmaceutically acceptable salt thereof.

In some embodiments, the HDAC inhibitor and the immunomodulatory drug are administered with a pharmaceutically acceptable carrier.

In some embodiments, the HDAC inhibitor and the immunomodulatory drug are administered in separate dosage forms. In other embodiments, the HDAC inhibitor and the immunomodulatory drug are administered in a single dosage form.

In some embodiments, the HDAC inhibitor and the immunomodulatory drug are administered at different times. In other embodiments, the HDAC inhibitor and the immunomodulatory drug are administered at substantially the same time.

In some embodiments, the combination of a HDAC inhibitor and an IMiD achieves a synergistic effect in the treatment of the subject in need thereof.

In some embodiments of the combinations and/or methods, the HDAC6 specific inhibitor is a compound of Formula I:

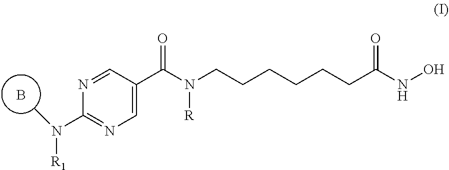

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl; and

the immunomodulatory drug is a compound of Formula III:

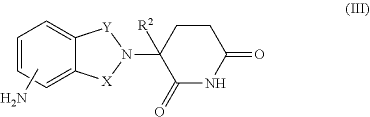

or a pharmaceutically acceptable salt thereof,
wherein,
one of X and Y is C═O, the other of X and Y is CH₂ or C═O; and
R² is H or C₁₋₆-alkyl.

In specific embodiments of the combinations and/or methods, the HDAC6 specific inhibitor is:

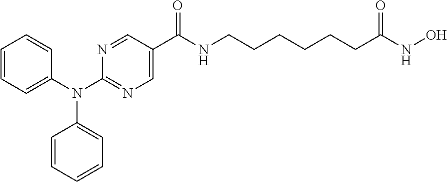 [SMILES: O=C(CCCCCCNC(=O)c1cnc(N(c2ccccc2)c2ccccc2)nc1)NO]

or a pharmaceutically acceptable salt thereof; and

the immunomodulatory drug is:

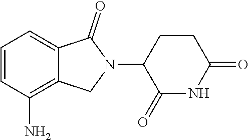 [SMILES: Nc1cccc2c1CN(C1CCC(=O)NC1=O)C2=O]

or a pharmaceutically acceptable salt thereof.

In specific embodiments of the combinations and/or methods, the HDAC6 specific inhibitor is:

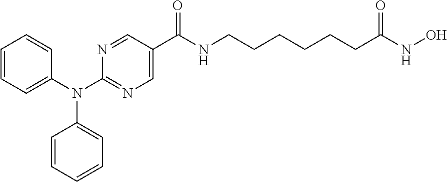 [SMILES: O=C(CCCCCCNC(=O)c1cnc(N(c2ccccc2)c2ccccc2)nc1)NO]

or a pharmaceutically acceptable salt thereof; and

the immunomodulatory drug is:

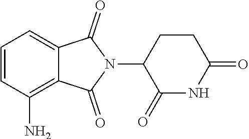 [SMILES: Nc1cccc2c1C(=O)N(C1CCC(=O)NC1=O)C2=O]

or a pharmaceutically acceptable salt thereof.

In specific embodiments of the combinations and/or methods, the HDAC6 specific inhibitor is:

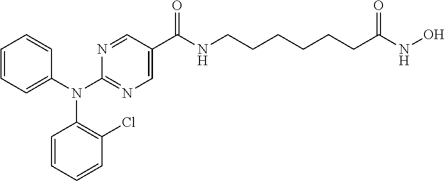 [SMILES: O=C(CCCCCCNC(=O)c1cnc(N(c2ccccc2)c2ccccc2Cl)nc1)NO]

or a pharmaceutically acceptable salt thereof; and

the immunomodulatory drug is:

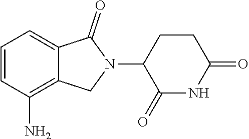 [SMILES: Nc1cccc2c1CN(C1CCC(=O)NC1=O)C2=O]

or a pharmaceutically acceptable salt thereof.

In specific embodiments of the combinations and/or methods, the HDAC6 specific inhibitor is:

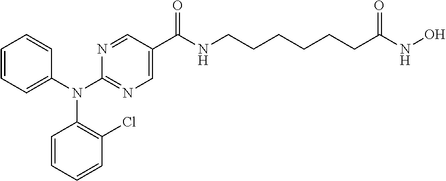 [SMILES: O=C(CCCCCCNC(=O)c1cnc(N(c2ccccc2)c2ccccc2Cl)nc1)NO]

or a pharmaceutically acceptable salt thereof; and

the immunomodulatory drug is:

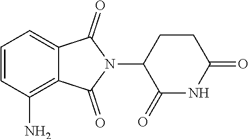 [SMILES: Nc1cccc2c1C(=O)N(C1CCC(=O)NC1=O)C2=O]

or a pharmaceutically acceptable salt thereof.

In some embodiments of the combinations and/or methods, the HDAC6 specific inhibitor is a compound of Formula II:

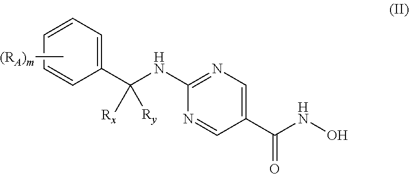

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂;

and
m is 0, 1, or 2; and

the immunomodulatory drug is a compound of Formula III:

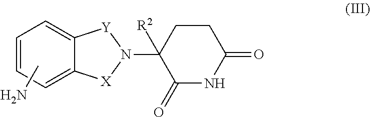

or a pharmaceutically acceptable salt thereof,
wherein,
one of X and Y is C═O, the other of X and Y is CH₂ or C═O; and
R² is H or C₁₋₆-alkyl.

In specific embodiments of the combinations and/or methods, the HDAC6 specific inhibitor is:

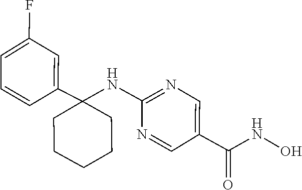 [SMILES: O=C(NO)c1cnc(NC2(c3cccc(F)c3)CCCCC2)nc1]

or a pharmaceutically acceptable salt thereof; and

the immunomodulatory drug is:

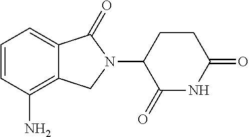 [SMILES: Nc1cccc2c1CN(C1CCC(=O)NC1=O)C2=O]

or a pharmaceutically acceptable salt thereof.

In specific embodiments of the combinations and/or methods, the HDAC6 specific inhibitor is:

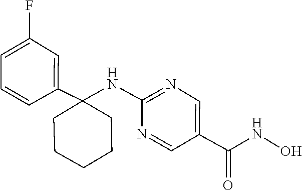 [SMILES: O=C(NO)c1cnc(NC2(c3cccc(F)c3)CCCCC2)nc1]

or a pharmaceutically acceptable salt thereof; and

the immunomodulatory drug is:

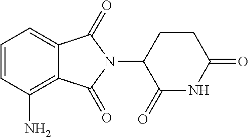 [SMILES: Nc1cccc2c1C(=O)N(C1CCC(=O)NC1=O)C2=O]

or a pharmaceutically acceptable salt thereof.

In specific embodiments of the combinations and/or methods, the HDAC6 specific inhibitor is:

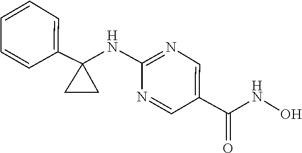 [SMILES: O=C(NO)c1cnc(NC2(c3ccccc3)CC2)nc1]

or a pharmaceutically acceptable salt thereof; and

the immunomodulatory drug is:

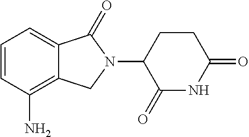 [SMILES: Nc1cccc2c1CN(C1CCC(=O)NC1=O)C2=O]

or a pharmaceutically acceptable salt thereof.

In specific embodiments of the combinations and/or methods, the HDAC6 specific inhibitor is:

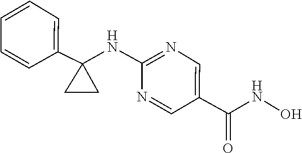 [SMILES: O=C(NO)c1cnc(NC2(c3ccccc3)CC2)nc1]

or a pharmaceutically acceptable salt thereof; and

the immunomodulatory drug is:

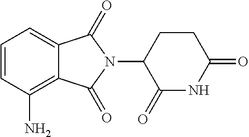 [SMILES: Nc1cccc2c1C(=O)N(C1CCC(=O)NC1=O)C2=O]

or a pharmaceutically acceptable salt thereof.

In some embodiments of the combinations and/or methods, the combinations can, optionally, further comprise an anti-inflammatory agent. In specific embodiments, the anti-inflammatory agent is dexamethasone.

In some embodiments of the combinations and/or methods, the HDAC6 specific inhibitor is a compound of Formula I:

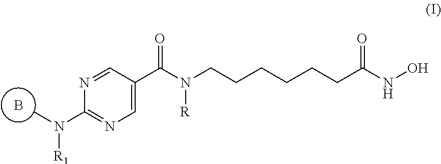

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and

R is H or C₁₋₆-alkyl;

the immunomodulatory drug is a compound of Formula III:

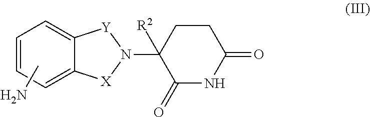

or a pharmaceutically acceptable salt thereof,
wherein,
one of X and Y is C═O, the other of X and Y is CH₂ or C═O; and
R² is H or C₁₋₆-alkyl; and

the anti-inflammatory agent is any anti-inflammatory agent.

In specific embodiments of the combinations and/or methods, the HDAC6 specific inhibitor is:

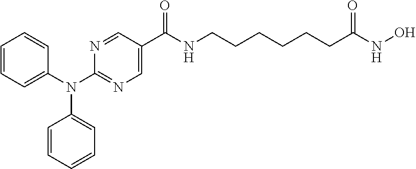 [SMILES: O=C(CCCCCCNC(=O)c1cnc(N(c2ccccc2)c2ccccc2)nc1)NO]

or a pharmaceutically acceptable salt thereof;

the immunomodulatory drug is:

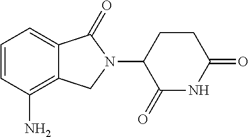 [SMILES: Nc1cccc2c1CN(C1CCC(=O)NC1=O)C2=O]

or a pharmaceutically acceptable salt thereof; and

the anti-inflammatory agent is dexamethasone.

In specific embodiments of the combinations and/or methods, the HDAC6 specific inhibitor is:

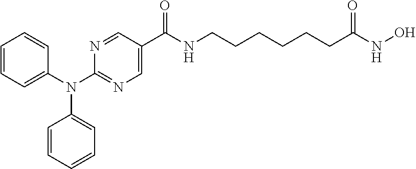 [SMILES: O=C(CCCCCCNC(=O)c1cnc(N(c2ccccc2)c2ccccc2)nc1)NO]

or a pharmaceutically acceptable salt thereof;

the immunomodulatory drug is:

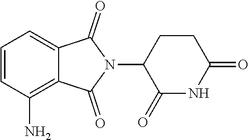 [SMILES: Nc1cccc2c1C(=O)N(C1CCC(=O)NC1=O)C2=O]

or a pharmaceutically acceptable salt thereof; and

the anti-inflammatory agent is dexamethasone.

In specific embodiments of the combinations and/or methods, the HDAC6 specific inhibitor is:

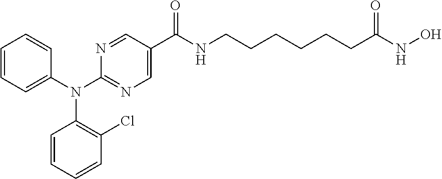 [SMILES: O=C(CCCCCCNC(=O)c1cnc(N(c2ccccc2)c2ccccc2Cl)nc1)NO]

or a pharmaceutically acceptable salt thereof;

the immunomodulatory drug is:

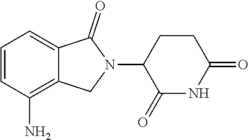 [SMILES: Nc1cccc2c1CN(C1CCC(=O)NC1=O)C2=O]

or a pharmaceutically acceptable salt thereof; and

the anti-inflammatory agent is dexamethasone.

In specific embodiments of the combinations and/or methods, the HDAC6 specific inhibitor is:

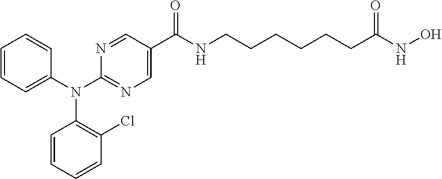 [SMILES: O=C(CCCCCCNC(=O)c1cnc(N(c2ccccc2)c2ccccc2Cl)nc1)NO]

or a pharmaceutically acceptable salt thereof;

the immunomodulatory drug is:

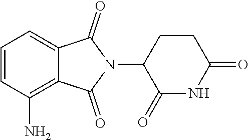 [SMILES: Nc1cccc2c1C(=O)N(C1CCC(=O)NC1=O)C2=O]

or a pharmaceutically acceptable salt thereof; and

the anti-inflammatory agent is dexamethasone.

In some embodiments of the combinations and/or methods, the HDAC6 specific inhibitor is a compound of Formula II:

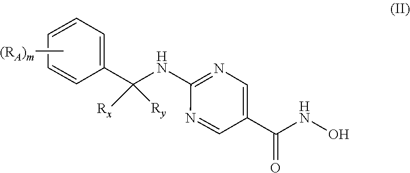

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, CN, or —NH₂;

and
m is 0, 1, or 2;

the immunomodulatory drug is a compound of Formula III:

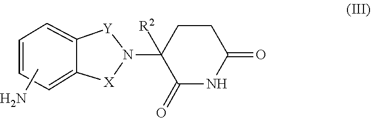

or a pharmaceutically acceptable salt thereof,
wherein,
one of X and Y is C═O, the other of X and Y is CH₂ or C═O; and
R² is H or C₁₋₆-alkyl; and

the anti-inflammatory agent is any anti-inflammatory agent.

In specific embodiments of the combinations and/or methods, the HDAC6 specific inhibitor is:

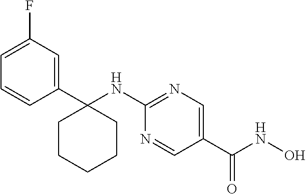 [SMILES: O=C(NO)c1cnc(NC2(c3cccc(F)c3)CCCCC2)nc1]

or a pharmaceutically acceptable salt thereof;

the immunomodulatory drug is:

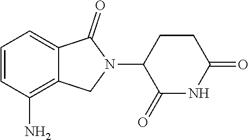 [SMILES: Nc1cccc2c1CN(C1CCC(=O)NC1=O)C2=O]

or a pharmaceutically acceptable salt thereof; and

the anti-inflammatory agent is dexamethasone.

In specific embodiments of the combinations and/or methods, the HDAC6 specific inhibitor is:

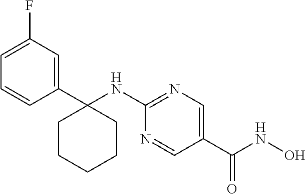 [SMILES: O=C(NO)c1cnc(NC2(c3cccc(F)c3)CCCCC2)nc1]

or a pharmaceutically acceptable salt thereof;

the immunomodulatory drug is:

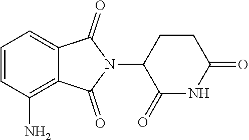 [SMILES: Nc1cccc2c1C(=O)N(C1CCC(=O)NC1=O)C2=O]

or a pharmaceutically acceptable salt thereof; and

the anti-inflammatory agent is dexamethasone.

In specific embodiments of the combinations and/or methods, the HDAC6 specific inhibitor is:

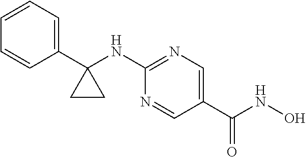 [SMILES: O=C(NO)c1cnc(NC2(c3ccccc3)CC2)nc1]

or a pharmaceutically acceptable salt thereof;

the immunomodulatory drug is:

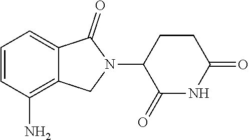 [SMILES: Nc1cccc2c1CN(C1CCC(=O)NC1=O)C2=O]

or a pharmaceutically acceptable salt thereof; and

the anti-inflammatory agent is dexamethasone.

In specific embodiments of the combinations and/or methods, the HDAC6 specific inhibitor is:

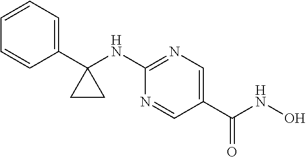 [SMILES: O=C(NO)c1cnc(NC2(c3ccccc3)CC2)nc1]

or a pharmaceutically acceptable salt thereof;

the immunomodulatory drug is:

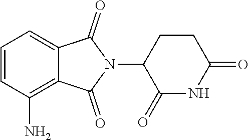 [SMILES: Nc1cccc2c1C(=O)N(C1CCC(=O)NC1=O)C2=O]

or a pharmaceutically acceptable salt thereof; and

the anti-inflammatory agent is dexamethasone.

In some embodiments, the HDAC inhibitor, the immunomodulatory drug, and the anti-inflammatory agent are administered with a pharmaceutically acceptable carrier.

In some embodiments, the HDAC inhibitor, the immunomodulatory drug, and the anti-inflammatory agent are administered in separate dosage forms. In other embodiments, the HDAC inhibitor, the immunomodulatory drug, and the anti-inflammatory agent are administered in a single dosage form.

In some embodiments, the HDAC inhibitor, the immunomodulatory drug, and the anti-inflammatory agent are administered at different times. In other embodiments, the HDAC inhibitor, the immunomodulatory drug, and the anti-inflammatory agent are administered at substantially the same time.

In a some embodiments, the HDAC inhibitor, the immunomodulatory drug, and the anti-inflammatory agent are present in amounts that produce a synergistic effect in the treatment of multiple myeloma in a subject in need thereof.

In some embodiments, the subject may have been previously treated with lenalidomide or bortezomib, or a combination thereof.

An embodiment of the invention includes a method for decreasing cell viability of cancer cells by administering a histone deacetylase (HDAC) specific inhibitor and an immunomodulatory drug (IMiD).

An embodiment of the invention includes a method for synergistically increasing apoptosis of cancer cells by administering a histone deacetylase (HDAC) specific inhibitor and an immunomodulatory drug (IMiD).

An embodiment of the invention includes a method for decreasing cell proliferation of cancer cells by administering a histone deacetylase (HDAC) specific inhibitor and an immunomodulatory drug (IMiD).

An embodiment of the invention includes a method for decreasing MYC and IRF4 expression in cancer cells by administering a histone deacetylase (HDAC) specific inhibitor and an immunomodulatory drug (IMiD).

An embodiment of the invention includes a method for increasing P21 expression in cancer cells by administering a histone deacetylase (HDAC) specific inhibitor and an immunomodulatory drug (IMiD).

Other objects, features, and advantages will become apparent from the following detailed description. The detailed description and specific examples are given for illustration only because various changes and modifications within the spirit and scope of the invention will become apparent to those skilled in the art from this detailed description. Further, the examples demonstrate the principle of the invention.

### BRIEF DESCRIPTION OF THE FIGURES

FIG. 1 is a graph that shows that Compound A enhances the activity of lenalidomide (Compound E).

FIG. 2 is a graph that shows that Compound A enhances the activity of pomalidomide (Compound F).

FIG. 3 is a graph that shows that Compound A enhances the activity of lenalidomide (Compound E) in the presence or absence of dexamethasone.

FIGS. 4A-C show the F_{A}/CI Synergy Plots after treatment of MM.1s cells with an HDAC6 inhibitor and an IMiD. FIG. 4A shows the F_{A}/CI Synergy Plots after treatment of MM.1s cells with Compound A, and either lenalidomide (top) or pomalidomide (bottom). FIG. 4B shows the F_{A}/CI Synergy Plots after treatment of MM.1s cells with Compound B, and either lenalidomide (top) or pomalidomide (bottom). FIG. 4C shows the F_{A}/CI Synergy Plots after treatment of MM.1s cells with Compound C, and either lenalidomide (top) or pomalidomide (bottom). Data points with CI values <1 indicate treatment combinations resulting in synergistic decreases in cellular viability.

FIGS. 5A-C show the F_{A}/CI Synergy Plots after treatment of H929 cells with an HDAC6 inhibitor and an IMiD. FIG. 5A shows the F_{A}/CI Synergy Plots after treatment of H929 cells with Compound A, and either lenalidomide (top) or pomalidomide (bottom). FIG. 5B shows the F_{A}/CI Synergy Plots after treatment of H929 cells with Compound B, and either lenalidomide (top) or pomalidomide (bottom). FIG. 5C shows the F_{A}/CI Synergy Plots after treatment of H929 cells with Compound C, and either lenalidomide (top) or pomalidomide (bottom). Data points with CI values <1 indicate treatment combinations resulting in synergistic decreases in cellular viability.

FIGS. 6A-B are a pair of graphs that show increased apoptosis in H929 cells treated with Compound A and an IMiD. FIG. 6A is a graph that shows apoptosis in H929 cells with Compound A and lenalidomide. FIG. 6B is a graph that shows apoptosis in H929 cells with Compound A and pomalidomide.

FIG. 7A is a graph that shows inhibition of MM.1s xenograft tumor growth with various combinations of Compound A, lenalidomide, and/or dexamethasone.

FIG. 7B is a graph that shows increased overall survival upon treatment of mice carrying H929 tumor xenografts with the combination of Compound B and pomalidomide relative to either single agent.

FIGS. 8A-C is a set of photographs of gels that show that the combination of Compound A, lenalidomide (Compound E), and dexamethasone leads to suppression of Myc expression, a key transcriptional regulator in cancer. Markers of apoptosis (cleaved PARP and caspase) are increased, and suppressors of apoptosis, such as XIAP, are decreased.

FIG. 8D is an image of an immunoblot from MM1s cells showing that the combination of Compound B and pomalidomide (Compound F) also leads to suppression of Myc expression. Markers of apoptosis (cleaved PARP and caspase) are increased, and suppressors of apoptosis, such as XIAP, are decreased by combination treatment.

FIGS. 9A-D are sets of F_{A}/CI Synergy Plots showing that the combination of HDAC6 inhibitors and IMiDs results in synergistic decreases in myeloma cell growth and viability. FIG. 9A is a set of graphs that show the results of experiments in which H929 myeloma cells were exposed to increasing doses of Compound A in combination with lenalidomide (top panel) or pomalidomide (bottom panel) at constant ratios. FIG. 9B is a set of graphs that show the results of experiments in which H929 myeloma cells were exposed to increasing doses of Compound C in combination with lenalidomide (top panel) or pomalidomide (bottom panel) at constant ratios. FIG. 9C is a set of graphs that show the results of experiments in which MM.1s myeloma cells were exposed to increasing doses of Compound A in combination with lenalidomide (top panel) or pomalidomide (bottom panel) at constant ratios. FIG. 9D is a set of graphs that show the results of experiments in which MM.1s myeloma cells were exposed to increasing doses of Compound C in combination with lenalidomide (top panel) or pomalidomide (bottom panel) at constant ratios.

FIGS. 9E-F are sets of graphs showing that the combination of HDAC6 inhibitors and IMiDs resulted in synergistic decreases in myeloma cell growth and viability. FIG. 9E shows the results of experiments in which H929 myeloma cells were exposed to increasing doses of Compound B in combination with lenalidomide (top panel) or pomalidomide (bottom panel) at constant ratios. FIG. 9F shows the results of experiments in which MM.1s myeloma cells were exposed to increasing doses of Compound B in combination with lenalidomide (top panel) or pomalidomide (bottom panel) at constant ratios. The combination index (CI) values for each dose combination are shown (Actual), as well as a simulation of CI values across the entire dosing range. Data points with CI values <1 indicate treatment combinations resulting in synergistic decreases in cellular viability.

FIGS. 10A-D are a series of graphs showing that combination treatment of multiple myeloma cells with Compound A and/or IMiDs results in decreased cell cycle progression relative to either single agent. FIG. 10A is a graph showing the effects of treatment of H929 myeloma cells for 3 days with DMSO, Compound A (2 μM), Lenalidomide (2 μM), Pomalidomide (1 μM), or combinations of Compound A with either IMiD on cell cycle inhibition. FIG. 10B is a graph showing the effects of treatment of H929 myeloma cells for 5 days with DMSO, Compound A (2 μM), Lenalidomide (2 μM), Pomalidomide (1 μM), or combinations of Compound A with either IMiD on cell cycle inhibition. FIG. 10C is a graph showing the effects of treatment of MM.1s myeloma cells for 3 days with DMSO, Compound A (2 μM), Lenalidomide (2 μM), Pomalidomide (1 μM), or combinations of Compound A with either IMiD on cell cycle inhibition. FIG. 10D is a graph showing the effects of treatment of MM.1s myeloma cells for 5 days with DMSO, Compound A (2 μM), Lenalidomide (2 μM), Pomalidomide (1 μM), or combinations of Compound A with either IMiD on cell cycle inhibition.

FIGS. 10E-F are graphs showing that combination treatment of multiple myeloma cells with Compound B and/or IMiDs resulted in decreased cell cycle progression relative to either single agent. FIG. 10E shows the effect of treatment of H929 myeloma cells for 4 days with DMSO, Compound B (2 μM), Lenalidomide (2 μM), Pomalidomide (1 μM), or combinations of Compound B with either IMiD on cell cycle inhibition. FIG. 10F show the effects of treatment of MM.1s myeloma cells for 5 days with DMSO, Compound B (2 μM), Lenalidomide (2 μM), Pomalidomide (1 μM), or combinations of Compound B with either IMiD on cell cycle inhibition.

FIGS. 11A-D are a series of graphs showing that combination treatment of multiple myeloma cells with Compound A and IMiDs results in synergistic increases in cellular apoptosis. FIG. 11A is a graph showing the effects of treatment of H929 myeloma cells for 5 days with DMSO, Compound A (2 μM), Lenalidomide (2 μM), Pomalidomide (1 μM), or combinations of Compound A with either IMiD on the induction of apoptosis. FIG. 11B is a graph showing the effects of treatment of H929 myeloma cells for 7 days with DMSO, Compound A (2 μM), Lenalidomide (2 μM), Pomalidomide (1 μM), or combinations of Compound A with either IMiD on the induction of apoptosis. FIG. 11C is a graph showing the effects of treatment of MM.1s myeloma cells for 5 days with DMSO, Compound A (2 μM), Lenalidomide (2 μM), Pomalidomide (1 μM), or combinations of Compound A with either IMiD on the induction of apoptosis. FIG. 11D is a graph showing the effects of treatment of MM.1s myeloma cells for 7 days with DMSO, Compound A (2 μM), Lenalidomide (2 μM), Pomalidomide (1 μM), or combinations of Compound A with either IMiD on the induction of apoptosis.

FIGS. 11E-F are graphs showing that treatment of multiple myeloma cells with Compound B and IMiDs results in synergistic increases in cellular apoptosis. FIG. 11E shows the effect of treatment of H929 myeloma cells for 4 days with DMSO, Compound B (2 μM), Lenalidomide (2 μM), Pomalidomide (1 μM), or combinations of Compound B with either IMiD on the induction of apoptosis. FIG. 11F shows the effect of treatment of MM.1s myeloma cells for 5 days with DMSO, Compound B (2 μM), Lenalidomide (2 μM), Pomalidomide (1 μM), or combinations of Compound B with either IMiD on the induction of apoptosis.

FIGS. 12A-E are a series of graphs showing that the mRNA expression level of MYC, IRF4, and CRBN are decreased by combination treatment with Compound A and IMiDs. FIG. 12A is a graph showing the effects of treatment of H929 myeloma cells with DMSO, Compound A (2 μM), Lenalidomide (1 μM), Pomalidomide (1 μM), or combinations of Compound A with either IMiD on the expression of MYC. FIG. 12B is a graph showing the effects of treatment of H929 myeloma cells with DMSO, Compound A (2 μM), Lenalidomide (1 μM), Pomalidomide (1 μM), or combinations of Compound A with either IMiD on the expression of IRF4. FIG. 12C is a graph showing the effects of treatment of H929 myeloma cells with DMSO, Compound A (2 μM), Lenalidomide (1 μM), Pomalidomide (1 μM), or combinations of Compound A with either IMiD on the expression of CRBN. FIG. 12D is a graph showing the effects of treatment of H929 myeloma cells with DMSO, Compound A (2 μM), Lenalidomide (1 μM), Pomalidomide (1 μM), or combinations of Compound A with either IMiD on the expression of P21. FIG. 12E is an immunoblot confirming, at the protein level in H929 cells after 48 hours of combination treatment, the reduction of MYC and IRF4 and the increase of P21 expression relative to any of the single agents.

FIG. 12F is an image of an immunoblot confirming, at the protein level in H929 cells, the reduction of IRF4 after 48 hours of combination treatment with Compound B and either lenalidomide or pomalidomide relative to any of the single agents.

FIG. 13A is a graph showing the effects of treatment of SCID-beige mice with Vehicle, Compound A alone, lenalidomide plus dexamethasone, or the triple combination of lenalidomide, dexamethasone, and Compound A.

FIG. 13B is a graph showing the effects of treatment with Vehicle, Compound B alone, pomalidomide alone, or the combination of pomalidomide and Compound B on the body weight of CB17-SCID mice. All combination treatments were well tolerated with no overt evidence of toxicity.

### DETAILED DESCRIPTION

The instant application is directed, generally, to combinations comprising a histone deacetylase (HDAC) inhibitor and an immunomodulatory drug (IMiD), and methods for the treatment of multiple myeloma. The combinations and/or methods may, optionally, further comprise an anti-inflammatory agent, such as dexamethasone.

### Definitions

Listed below are definitions of various terms used to describe this invention. These definitions apply to the terms as they are used throughout this specification and claims, unless otherwise limited in specific instances, either individually or as part of a larger group.

The term “about” generally indicates a possible variation of no more than 10%, 5%, or 1% of a value. For example, “about 25 mg/kg” will generally indicate, in its broadest sense, a value of 22.5-27.5 mg/kg, i.e., 25±2.5 mg/kg.

The term “alkyl” refers to saturated, straight- or branched-chain hydrocarbon moieties containing, in certain embodiments, between one and six, or one and eight carbon atoms, respectively. Examples of C₁₋₆ alkyl moieties include, but are not limited to, methyl, ethyl, propyl, isopropyl, n-butyl, tent-butyl, neopentyl, n-hexyl moieties; and examples of C₁₋₈ alkyl moieties include, but are not limited to, methyl, ethyl, propyl, isopropyl, n-butyl, tent-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

The term “alkoxy” refers to an —O-alkyl moiety.

The terms “cycloalkyl” or “cycloalkylene” denote a monovalent group derived from a monocyclic or polycyclic saturated or partially unsatured carbocyclic ring compound. Examples of C₃-C₈-cycloalkyl include, but are not limited to, cyclopropyl, cyclobutyl, cyclopentyl, cyclohexyl, cycloheptyl and cyclooctyl; and examples of C₃-C₁₂-cycloalkyl include, but are not limited to, cyclopropyl, cyclobutyl, cyclopentyl, cyclohexyl, bicyclo [2.2.1] heptyl, and bicyclo [2.2.2] octyl. Also contemplated are monovalent groups derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “aryl” refers to a mono- or poly-cyclic carbocyclic ring system having one or more aromatic rings, fused or non-fused, including, but not limited to, phenyl, naphthyl, tetrahydronaphthyl, indanyl, idenyl and the like. In some embodiments, aryl groups have 6 carbon atoms. In some embodiments, aryl groups have from six to ten carbon atoms. In some embodiments, aryl groups have from six to sixteen carbon atoms.

The term “combination” refers to two or more therapeutic agents to treat a therapeutic condition or disorder described in the present disclosure. Such combination of therapeutic agensts may be in the form of a single pill, capsule, or intravenous solution. However, the term “combination” also encompasses the situation when the two or more therapeutic agents are in separate pills, capsules, or intravenous solutions. Likewise, the term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, or in separate containers (e.g., capsules) for each active ingredient. In addition, such administration also encompasses use of each type of therapeutic agent in a sequential manner, either at approximately the same time or at different times. In either case, the treatment regimen will provide beneficial effects of the drug combination in treating the conditions or disorders described herein.

The term “heteroaryl” refers to a mono- or poly-cyclic (e.g., bi-, or tri-cyclic or more) fused or non-fused moiety or ring system having at least one aromatic ring, where one or more of the ring-forming atoms is a heteroatom such as oxygen, sulfur, or nitrogen. In some embodiments, the heteroaryl group has from about one to six carbon atoms, and in further embodiments from one to fifteen carbon atoms. In some embodiments, the heteroaryl group contains five to sixteen ring atoms of which one ring atom is selected from oxygen, sulfur, and nitrogen; zero, one, two, or three ring atoms are additional heteroatoms independently selected from oxygen, sulfur, and nitrogen; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiazolyl, thiadiazolyl, oxadiazolyl, thiophenyl, furanyl, indolyl, quinolinyl, isoquinolinyl, benzimidazolyl, benzooxazolyl, quinoxalinyl, acridinyl, and the like.

The term “halo” refers to a halogen, such as fluorine, chlorine, bromine, and iodine.

The term “HDAC” refers to histone deacetylases, which are enzymes that remove the acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hda1 gene. Class III HDACs, which are also known as the sirtuins are related to the Sir2 gene and include SIRT1-7. Class IV HDACs, which contains only HDAC11, has features of both Class I and II HDACs. The term “HDAC” refers to any one or more of the 18 known histone deacetylases, unless otherwise specified.

The term “HDAC6 specific” means that the compound binds to HDAC6 to a substantially greater extent, such as 5×, 10×, 15×, 20× greater or more, than to any other type of HDAC enzyme, such as HDAC1 or HDAC2. That is, the compound is selective for HDAC6 over any other type of HDAC enzyme. For example, a compound that binds to HDAC6 with an IC₅₀ of 10 nM and to HDAC1 with an IC₅₀ of 50 nM is HDAC6 specific. On the other hand, a compound that binds to HDAC6 with an IC₅₀ of 50 nM and to HDAC1 with an IC₅₀ of 60 nM is not HDAC6 specific

The term “inhibitor” is synonymous with the term antagonist.

### Histone Deacetylase (HDAC) Inhibitors

Provided herein are pharmaceutical combinations for the treatment of multiple myeloma in a subject in need thereof Also provided herein are methods for treating multiple myeloma in a subject in need thereof.

The combinations and methods of the invention comprise a histone deacetylase (HDAC) inhibitor. The HDAC inhibitor may be any HDAC inhibitor. Thus, the HDAC inhibitor may be selective or non-selective to a particular type of histone deacetylase enzyme. Preferably, the HDAC inhibitor is a selective HDAC inhibitor. More preferably, the HDAC inhibitor is an HDAC6 inhibitor.

In some embodiments, the HDAC6 specific inhibitor is a compound of Formula I:

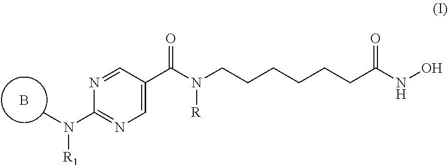

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl.

Representative compounds of Formula I include, but are not limited to:

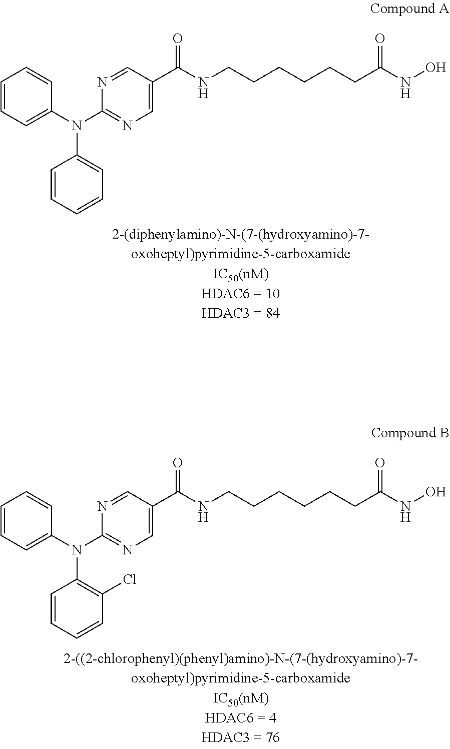 [SMILES: O=C(CCCCCCNC(=O)c1cnc(N(c2ccccc2)c2ccccc2)nc1)NO ; O=C(CCCCCCNC(=O)c1cnc(N(c2ccccc2)c2ccccc2Cl)nc1)NO]

or pharmaceutically acceptable salts thereof.

The preparation and properties of selective HDAC6 inhibitors according to Formula I are provided in International Patent Application No. PCT/US2011/021982, the entire contents of which is incorporated herein by reference.

In other embodiments, the HDAC6 specific inhibitor is a compound of Formula II:

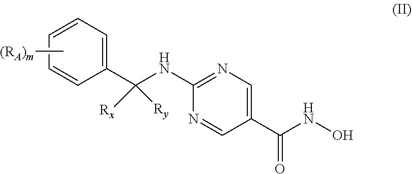

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂;

and
m is 0, 1, or 2.

Representative compounds of Formula II include, but are not limited to:

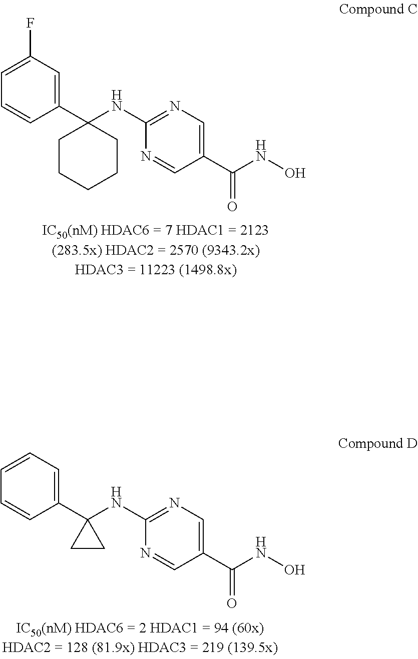 [SMILES: O=C(NO)c1cnc(NC2(c3cccc(F)c3)CCCCC2)nc1 ; O=C(NO)c1cnc(NC2(c3ccccc3)CC2)nc1]

or pharmaceutically acceptable salts thereof.

The preparation and properties of selective HDAC6 inhibitors according to Formula II are provided in International Patent Application No. PCT/US2011/060791, the entire contents of which are incorporated herein by reference.

In some embodiments, the compounds described herein are unsolvated. In other embodiments, one or more of the compounds are in solvated form. As known in the art, the solvate can be any of pharmaceutically acceptable solvent, such as water, ethanol, and the like.

### Immunomodulatory Drugs (IMiDs)

The combinations and methods of the invention comprise an immunomodulatory drug (IMiD). The IMiD may be any immunomodulatory drug. Preferably, the IMiD is a thalidomide of Formula III.

In some embodiments, the immunomodulatory drug is a compound of Formula III:

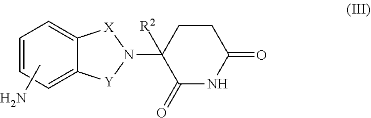

or a pharmaceutically acceptable salt thereof,
wherein,
one of X and Y is C═O, the other of X and Y is CH₂ or C═O; and
R² is H or C₁₋₆-alkyl.

Representative compounds of Formula III include, but are not limited to:

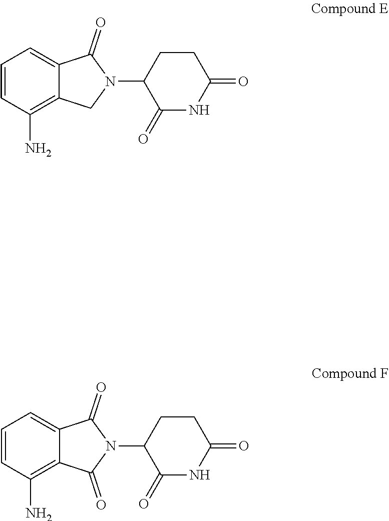 [SMILES: Nc1cccc2c1CN(C1CCC(=O)NC1=O)C2=O ; Nc1cccc2c1C(=O)N(C1CCC(=O)NC1=O)C2=O]

or pharmaceutically acceptable salts thereof.

The preparation and properties of the immunomodulatory drugs according to Formula III are provided in U.S. Pat. Nos. 5,635,517; 6,281,230; 6,335,349; and 6,476,052; as well as International Patent Application No. PCT/US97/013375, each of which is incorporated herein by reference in its entirety.

In some embodiments, the compounds described herein are unsolvated. In other embodiments, one or more of the compounds are in solvated form. As known in the art, the solvate can be any of pharmaceutically acceptable solvent, such as water, ethanol, and the like.

### Anti-inflammatory Agents

The combinations and methods of the invention may, optionally, further comprise an anti-inflammatory agent. The anti-inflammatory agent may be any anti-inflammatory agent. Preferably, the anti-inflammatory agent is dexamethasone.

In some embodiments, the compounds described herein are unsolvated. In other embodiments, one or more of the compounds are in solvated form. As known in the art, the solvate can be any of pharmaceutically acceptable solvent, such as water, ethanol, and the like.

### Combinations/Pharmaceutical Combinations

Provided herein are combinations for the treatment of multiple myeloma in a subject in need thereof. Provided in some embodiments are combinations comprising a histone deacetylase (HDAC) inhibitor and an immunomodulatory drug (IMiD) for the treatment of multiple myeloma in a subject in need thereof. In some specific embodiments, the combinations do not include dexamethasone. In other specific embodiments, the combinations may, optionally, further comprise an anti-inflammatory agent, such as dexamethasone.

In some embodiments of the combinations, the HDAC inhibitor is an HDAC6 inhibitor. In specific embodiments, the HDAC6 specific inhibitor is a compound of Formula I:

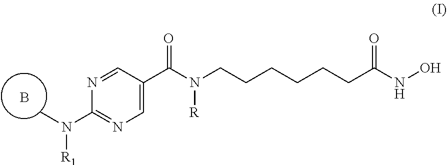

or a pharmaceutically acceptable salt thereof.

In preferred embodiments, the compound of Formula I is:

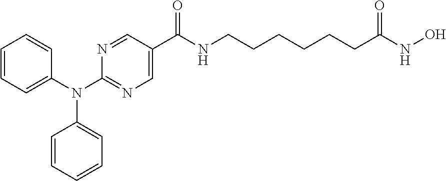 [SMILES: O=C(CCCCCCNC(=O)c1cnc(N(c2ccccc2)c2ccccc2)nc1)NO]

or a pharmaceutically acceptable salt thereof.

In yet other embodiments, the compound of Formula I is:

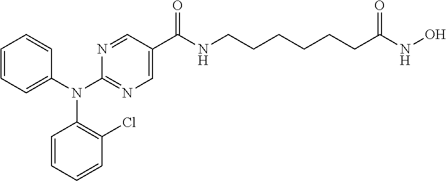 [SMILES: O=C(CCCCCCNC(=O)c1cnc(N(c2ccccc2)c2ccccc2Cl)nc1)NO]

or a pharmaceutically acceptable salt thereof.

In other specific embodiments, the HDAC6 specific inhibitor is a compound of Formula II:

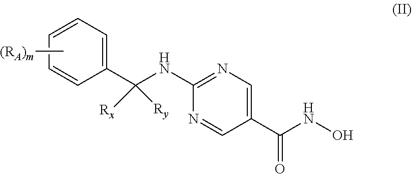

or a pharmaceutically acceptable salt thereof.

In preferred embodiments, the compound of Formula II is:

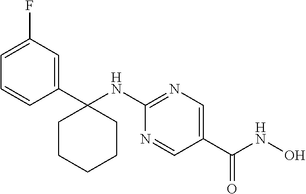 [SMILES: O=C(NO)c1cnc(NC2(c3cccc(F)c3)CCCCC2)nc1]

or a pharmaceutically acceptable salt thereof.

In other preferred embodiments, the compound of Formula II is:

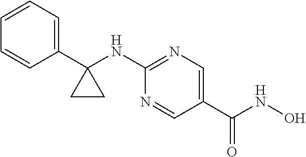 [SMILES: O=C(NO)c1cnc(NC2(c3ccccc3)CC2)nc1]

or a pharmaceutically acceptable salt thereof.

In some embodiments of the combinations, the immunomodulatory drug is a compound of Formula III:

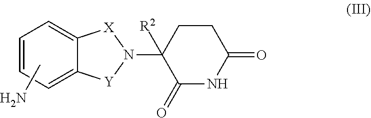

or a pharmaceutically acceptable salt thereof.

In preferred embodiments, the compound of Formula III is:

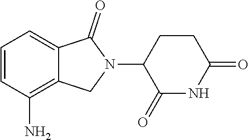 [SMILES: Nc1cccc2c1CN(C1CCC(=O)NC1=O)C2=O]

or a pharmaceutically acceptable salt thereof.

In yet other preferred embodiments, the compound of Formula III is:

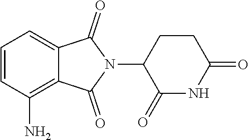 [SMILES: Nc1cccc2c1C(=O)N(C1CCC(=O)NC1=O)C2=O]

or a pharmaceutically acceptable salt thereof.

In one embodiment, provided herein is a combination therapy comprising an HDAC6 specific inhibitor and an immunomodulatory drug, wherein the HDAC6 specific inhibitor is a compound of Formula I:

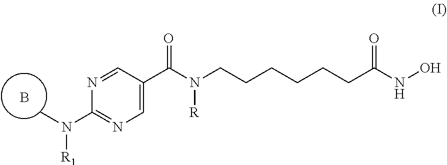

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl; and

the immunomodulatory drug is a compound of Formula III:

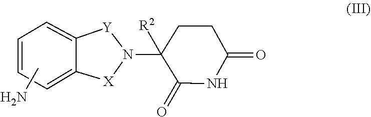

or a pharmaceutically acceptable salt thereof,
wherein,
one of X and Y is C═O, the other of X and Y is CH₂ or C═O; and
R² is H or C₁₋₆-alkyl.

As described in further detail below, some embodiments of this combination include an anti-inflammatory agent, while other embodiments of this combination do not include dexamethasone.

In specific embodiments of the combinations, the HDAC6 specific inhibitor is:

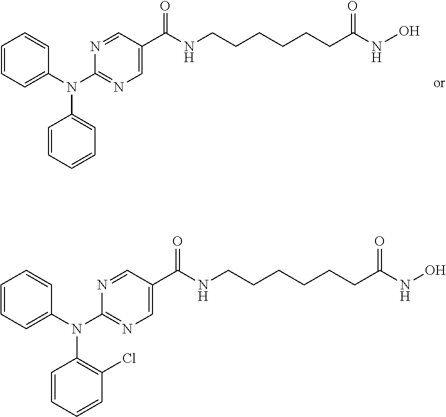 [SMILES: O=C(CCCCCCNC(=O)c1cnc(N(c2ccccc2)c2ccccc2)nc1)NO ; O=C(CCCCCCNC(=O)c1cnc(N(c2ccccc2)c2ccccc2Cl)nc1)NO]

or pharmaceutically acceptable salts thereof; and

the immunomodulatory drug is:

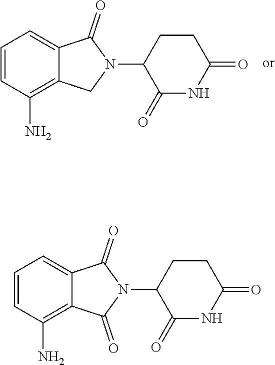 [SMILES: Nc1cccc2c1CN(C1CCC(=O)NC1=O)C2=O ; Nc1cccc2c1C(=O)N(C1CCC(=O)NC1=O)C2=O]

or a pharmaceutically acceptable salt thereof.

In some embodiments, when the combination includes Compound A and Compound E, the combination does not include dexamethasone. Similarly, when the combination includes Compound A and Compound F, some embodiments of the combination exclude dexamethasone. However, when the combination includes Compound A and Compound F, some embodiments of the combination include an anti-inflammatory agent, such as dexamethasone.

In another embodiment, provided herein is a combination therapy comprising an HDAC6 specific inhibitor and an immunomodulatory drug, wherein the HDAC6 specific inhibitor is a compound of Formula II:

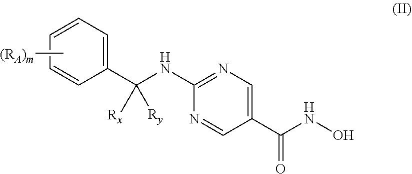

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂;

and
m is 0, 1, or 2; and

the immunomodulatory drug is a compound of Formula III:

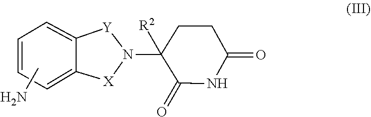

or a pharmaceutically acceptable salt thereof,
wherein,
one of X and Y is C═O, the other of X and Y is CH₂ or C═O; and
R² is H or C₁₋₆-alkyl.

In specific embodiments of the combinations, the HDAC6 specific inhibitor is:

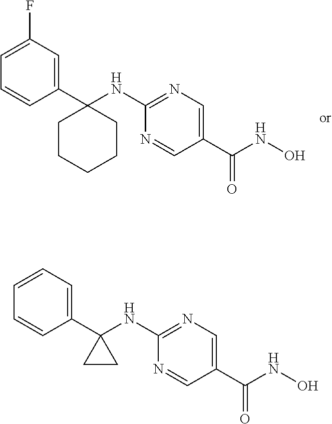 [SMILES: O=C(NO)c1cnc(NC2(c3cccc(F)c3)CCCCC2)nc1 ; O=C(NO)c1cnc(NC2(c3ccccc3)CC2)nc1]

or a pharmaceutically acceptable salt thereof; and

the immunomodulatory drug is:

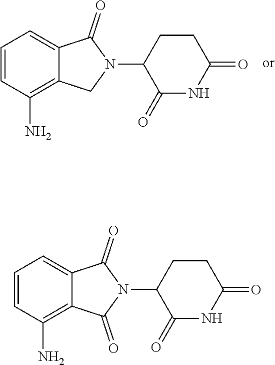 [SMILES: Nc1cccc2c1CN(C1CCC(=O)NC1=O)C2=O ; Nc1cccc2c1C(=O)N(C1CCC(=O)NC1=O)C2=O]

or a pharmaceutically acceptable salt thereof.

In some embodiments of the combinations, the combinations may, optionally, further comprise an anti-inflammatory agent. In specific embodiments, the anti-inflammatory agent is dexamethasone.

In one embodiment, provided herein is a combination therapy comprising an HDAC6 specific inhibitor, an immunomodulatory drug, and an anti-inflammatory agent, wherein the HDAC6 specific inhibitor is a compound of Formula I:

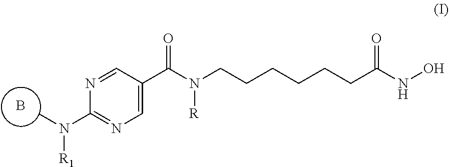

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl;

the immunomodulatory drug is a compound of Formula III:

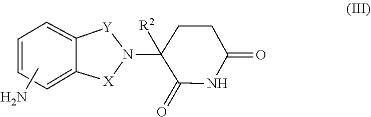

or a pharmaceutically acceptable salt thereof,
wherein,
one of X and Y is C═O, the other of X and Y is CH₂ or C═O; and
R² is H or C₁₋₆-alkyl; and

the anti-inflammatory agent is any anti-inflammatory agent.

In specific embodiments of the combinations, the HDAC6 specific inhibitor is:

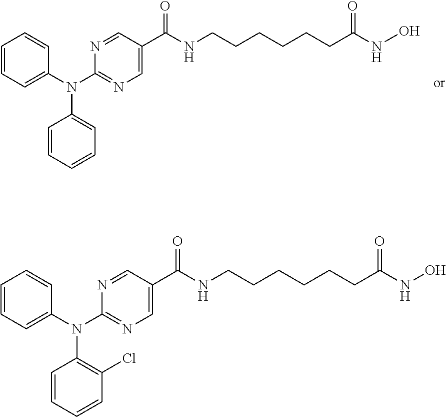 [SMILES: O=C(CCCCCCNC(=O)c1cnc(N(c2ccccc2)c2ccccc2)nc1)NO ; O=C(CCCCCCNC(=O)c1cnc(N(c2ccccc2)c2ccccc2Cl)nc1)NO]

or pharmaceutically acceptable salts thereof;

the immunomodulatory drug is:

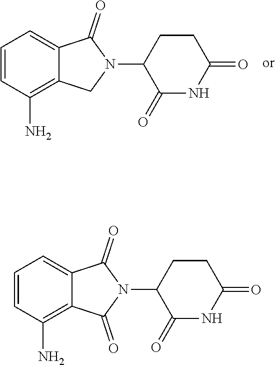 [SMILES: Nc1cccc2c1CN(C1CCC(=O)NC1=O)C2=O ; Nc1cccc2c1C(=O)N(C1CCC(=O)NC1=O)C2=O]

or pharmaceutically acceptable salts thereof; and

the anti-inflammatory agent is dexamethasone.

In another embodiment, provided herein is a combination therapy comprising an HDAC6 specific inhibitor, an immunomodulatory drug, and an anti-inflammatory agent, wherein the HDAC6 specific inhibitor is a compound of Formula II:

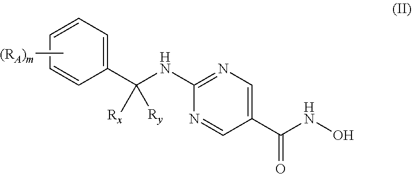

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂;

and
m is 0, 1, or 2;

the immunomodulatory drug is a compound of Formula III:

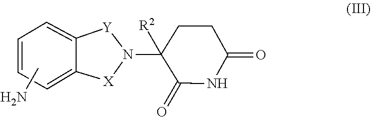

or a pharmaceutically acceptable salt thereof,
wherein,
one of X and Y is C═O, the other of X and Y is CH₂ or C═O; and

R² is H or C₁₋₆-alkyl; and

the anti-inflammatory agent is any anti-inflammatory agent.

In specific embodiments of the combinations, the HDAC6 specific inhibitor is:

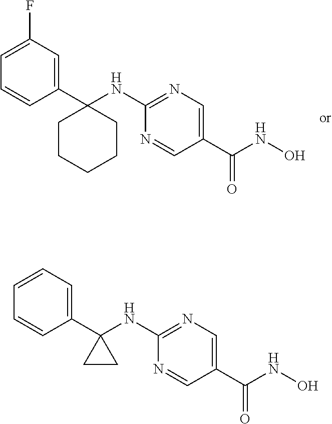 [SMILES: O=C(NO)c1cnc(NC2(c3cccc(F)c3)CCCCC2)nc1 ; O=C(NO)c1cnc(NC2(c3ccccc3)CC2)nc1]

or pharmaceutically acceptable salts thereof;

the immunomodulatory drug is:

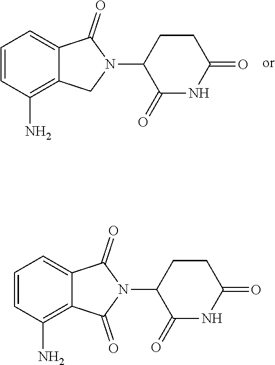 [SMILES: Nc1cccc2c1CN(C1CCC(=O)NC1=O)C2=O ; Nc1cccc2c1C(=O)N(C1CCC(=O)NC1=O)C2=O]

or pharmaceutically acceptable salts thereof; and

the anti-inflammatory agent is dexamethasone.

Although the compounds of Formulas I, II, and III are depicted in their neutral forms, in some embodiments, these compounds are used in a pharmaceutically acceptable salt form. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17.sup.th ed., Mack Publishing Company, Easton, Pa., 1985, p. 1418 and Journal of Pharmaceutical Science, 66, 2 (1977), each of which is incorporated herein by reference in its entirety.

### Administration/Dose

In some embodiments, the HDAC inhibitor (a compound of Formula I or II) is administered simultaneously with the immunomodulatory drug (a compound of Formula III). Simultaneous administration typically means that both compounds enter the patient at precisely the same time. However, simultaneous administration also includes the possibility that the HDAC inhibitor and the IMiD enter the patient at different times, but the difference in time is sufficiently miniscule that the first administered compound is not provided the time to take effect on the patient before entry of the second administered compound. Such delayed times typically correspond to less than 1 minute, and more typically, less than 30 seconds. In one example, wherein the compounds are in solution, simultaneous administration can be achieved by administering a solution containing the combination of compounds. In another example, simultaneous administration of separate solutions, one of which contains the HDAC inhibitor and the other of which contains the IMiD, can be employed. In one example wherein the compounds are in solid form, simultaneous administration can be achieved by administering a composition containing the combination of compounds. Alternatively, simultaneous administration can be achieved by administering two separate compositions, one comprising the HDAC inhibitor and the other comprising the IMiD.

In other embodiments, the HDAC inhibitor and the IMiD are not administered simultaneously. In some embodiments, the HDAC inhibitor is administered before the IMiD. In other embodiments, the IMiD is administered before the HDAC inhibitor. The time difference in non-simultaneous administrations can be greater than 1 minute, five minutes, 10 minutes, 15 minutes, 30 minutes, 45 minutes, 60 minutes, two hours, three hours, six hours, nine hours, 12 hours, 24 hours, 36 hours, or 48 hours. In other embodiments, the first administered compound is provided time to take effect on the patient before the second administered compound is administered. Generally, the difference in time does not extend beyond the time for the first administered compound to complete its effect in the patient, or beyond the time the first administered compound is completely or substantially eliminated or deactivated in the patient.

In some embodiments, one or both of the HDAC inhibitor and immunomodulatory drug are administered in a therapeutically effective amount or dosage. A “therapeutically effective amount” is an amount of HDAC6 inhibitor (a compound of Formula I or II) or an immunomodulatory drug (a compound of Formula III) that, when administered to a patient by itself, effectively treats the multiple myeloma. An amount that proves to be a “therapeutically effective amount” in a given instance, for a particular subject, may not be effective for 100% of subjects similarly treated for the disease or condition under consideration, even though such dosage is deemed a “therapeutically effective amount” by skilled practitioners. The amount of the compound that corresponds to a therapeutically effective amount is strongly dependent on the type of cancer, stage of the cancer, the age of the patient being treated, and other facts. In general, therapeutically effective amounts of these compounds are well-known in the art, such as provided in the supporting references cited above.

In other embodiments, one or both of the HDAC inhibitor and immunomodulatory drug are administered in a sub-therapeutically effective amount or dosage. A sub-therapeutically effective amount is an amount of HDAC inhibitor (a compound of Formula I or II) or an immunomodulatory drug (a compound of Formula III) that, when administered to a patient by itself, does not completely inhibit over time the biological activity of the intended target.

Whether administered in therapeutic or sub-therapeutic amounts, the combination of the HDAC inhibitor and the immunomodulatory drug should be effective in treating multiple myeloma. For example, a sub-therapeutic amount of a compound of Formula III (immunomodulatory drug) can be an effective amount if, when combined with a compound a compound of Formula I or II (HDAC inhibitor), the combination is effective in the treatment of multiple myeloma.

In some embodiments, the combination of compounds exhibits a synergistic effect (i.e., greater than additive effect) in the treatment of the multiple myeloma. The term “synergistic effect” refers to the action of two agents, such as, for example, a HDAC inhibitor and an IMiD, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In different embodiments, depending on the combination and the effective amounts used, the combination of compounds can inhibit cancer growth, achieve cancer stasis, or even achieve substantial or complete cancer regression.

While the amounts of a HDAC inhibitor and an IMiD should result in the effective treatment of multiple myeloma, the amounts, when combined, are preferably not excessively toxic to the patient (i.e., the amounts are preferably within toxicity limits as established by medical guidelines). In some embodiments, either to prevent excessive toxicity and/or provide a more efficacious treatment of multiple myeloma, a limitation on the total administered dosage is provided. Typically, the amounts considered herein are per day; however, half-day and two-day or three-day cycles also are considered herein.

Different dosage regimens may be used to treat multiple myeloma. In some embodiments, a daily dosage, such as any of the exemplary dosages described above, is administered once, twice, three times, or four times a day for three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

In some embodiments, each dosage contains both an HDAC inhibitor and an IMiD to be delivered as a single dosage, while in other embodiments, each dosage contains either a HDAC inhibitor and an IMiD to be delivered as separate dosages.

Compounds of Formula I, II, or III, or their pharmaceutically acceptable salts or solvate forms, in pure form or in an appropriate pharmaceutical composition, can be administered via any of the accepted modes of administration or agents known in the art. The compounds can be administered, for example, orally, nasally, parenterally (intravenous, intramuscular, or subcutaneous), topically, transdermally, intravaginally, intravesically, intracistemally, or rectally. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, preferably in unit dosage forms suitable for simple administration of precise dosages. A particular route of administration is oral, particularly one in which a convenient daily dosage regimen can be adjusted according to the degree of severity of the disease to be treated.

As discussed above, the HDAC inhibitor and the IMiD of the pharmaceutical combination can be administered in a single unit dose or separate dosage forms. Accordingly, the phrase “pharmaceutical combination” includes a combination of two drugs in either a single dosage form or a separate dosage forms, i.e., the pharmaceutically acceptable carriers and excipients described throughout the application can be combined with an HDAC inhibitor and an IMiD in a single unit dose, as well as individually combined with a HDAC inhibitor and an IMiD when these compounds are administered separately.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

Solid dosage forms can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the HDAC inhibitors or immmunomodulatory drugs described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Generally, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compound described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

### Methods of Treatment

The invention relates to methods for treating multiple myeloma in a subject in need thereof comprising administering to the subject a pharmaceutical combination of the invention. Thus, provided herein are methods for treating multiple myeloma in a subject in need thereof comprising administering to the subject a therapeutically effective amount of a combination comprising an HDAC inhibitor and an immunomodulatory drug. In specific embodiments of the methods, the combinations may, optionally, further comprise an anti-inflammatory agent, such as dexamethasone.

The subject considered herein is typically a human. However, the subject can be any mammal for which treatment is desired. Thus, the methods described herein can be applied to both human and veterinary applications.

The terms “treating” or “treatment” indicates that the method has, at the least, mitigated abnormal cellular proliferation. For example, the method can reduce the rate of myeloma growth in a patient, or prevent the continued growth or spread of the myeloma, or even reduce the overall reach of the myeloma.

As such, in one embodiment, provided herein is a method for treating multiple myeloma in a subject in need thereof comprising administering to the subject a therapeutically effective amount of Compound A and Compound E. The combination in this method does not include dexamethasone.

In another embodiment is a method for treating multiple myeloma in a subject in need thereof comprising administering to the subject a therapeutically effective amount of Compound A and Compound F. When the combination in this method includes Compound A and Compound F, some embodiments of the combination exclude dexamethasone. However, when the combination includes Compound A and Compound F, some embodiments of the combination include an anti-inflammatory agent, such as dexamethasone.

In another embodiment is a method for treating multiple myeloma in a subject in need thereof comprising administering to the subject a therapeutically effective amount of Compound B and Compound E. In some embodiments, this combination in this method does not include dexamethasone. However, in some embodiments, this combination includes an anti-inflammatory agent, such as dexamethasone.

In another embodiment is a method for treating multiple myeloma in a subject in need thereof comprising administering to the subject a therapeutically effective amount of Compound B and Compound F. In some embodiments, this combination in this method does not include dexamethasone. However, in some embodiments, this combination includes an anti-inflammatory agent, such as dexamethasone.

In another embodiment is a method for treating multiple myeloma in a subject in need thereof comprising administering to the subject a therapeutically effective amount of Compound C and Compound E.

In another embodiment is a method for treating multiple myeloma in a subject in need thereof comprising administering to the subject a therapeutically effective amount of Compound C and Compound F.

In another embodiment is a method for treating multiple myeloma in a subject in need thereof comprising administering to the subject a therapeutically effective amount of Compound D and Compound E.

In another embodiment is a method for treating multiple myeloma in a subject in need thereof comprising administering to the subject a therapeutically effective amount of Compound D and Compound F.

As stated previously, the methods may further comprise an anti-inflammatory agent.

In another embodiment is a method for treating multiple myeloma in a subject in need thereof comprising administering to the subject a therapeutically effective amount of Compound A, Compound F, and dexamethasone.

In another embodiment is a method for treating multiple myeloma in a subject in need thereof comprising administering to the subject a therapeutically effective amount of Compound B, Compound E, and dexamethasone.

In another embodiment is a method for treating multiple myeloma in a subject in need thereof comprising administering to the subject a therapeutically effective amount of Compound B, Compound F, and dexamethasone.

In another embodiment is a method for treating multiple myeloma in a subject in need thereof comprising administering to the subject a therapeutically effective amount of Compound C, Compound E, and dexamethasone.

In another embodiment is a method for treating multiple myeloma in a subject in need thereof comprising administering to the subject a therapeutically effective amount of Compound C, Compound F, and dexamethasone.

In another embodiment is a method for treating multiple myeloma in a subject in need thereof comprising administering to the subject a therapeutically effective amount of Compound D, Compound E, and dexamethasone.

In another embodiment is a method for treating multiple myeloma in a subject in need thereof comprising administering to the subject a therapeutically effective amount of Compound D, Compound F, and dexamethasone.

An embodiment of the invention includes a method for decreasing cell viability of cancer cells by administering a histone deacetylase (HDAC) specific inhibitor and an immunomodulatory drug (IMiD).

An embodiment of the invention includes a method for synergistically increasing apoptosis of cancer cells by administering a histone deacetylase (HDAC) specific inhibitor and an immunomodulatory drug (IMiD).

An embodiment of the invention includes a method for decreasing cell proliferation of cancer cells by administering a histone deacetylase (HDAC) specific inhibitor and an immunomodulatory drug (IMiD).

An embodiment of the invention includes a method for decreasing MYC and IRF4 expression in cancer cells by administering a histone deacetylase (HDAC) specific inhibitor and an immunomodulatory drug (IMiD).

An embodiment of the invention includes a method for increasing P21 expression in cancer cells by administering a histone deacetylase (HDAC) specific inhibitor and an immunomodulatory drug (IMiD).

### Kits

In other embodiments, kits are provided. Kits according to the invention include package(s) comprising compounds or compositions of the invention. In some embodiments, kits comprise a HDAC inhibitor, or a pharmaceutically acceptable salt thereof, and an IMiD or a pharmaceutically acceptable salt thereof.

The phrase “package” means any vessel containing compounds or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit can also contain items that are not contained within the package, but are attached to the outside of the package, for example, pipettes.

Kits can further contain instructions for administering compounds or compositions of the invention to a patient. Kits also can comprise instructions for approved uses of compounds herein by regulatory agencies, such as the United States Food and Drug Administration. Kits can also contain labeling or product inserts for the compounds. The package(s) and/or any product insert(s) may themselves be approved by regulatory agencies. The kits can include compounds in the solid phase or in a liquid phase (such as buffers provided) in a package. The kits can also include buffers for preparing solutions for conducting the methods, and pipettes for transferring liquids from one container to another.

### EXAMPLES

Examples have been set forth below for the purpose of illustration and to describe certain specific embodiments of the invention. However, the scope of the claims is not to be in any way limited by the examples set forth herein. Various changes and modifications to the disclosed embodiments will be apparent to those skilled in the art and such changes and modifications including, without limitation, those relating to the chemical structures, subtitutents, derivatives, formulations and/or methods of the invention may be made without departing from the spirit of the invention and the scope of the appended claims. Definitions of the variables in the structures in the schemes herein are commensurate with those of corresponding positions in the formulae presented herein.

The synthesis of the compounds of Formula I is provided in PCT/US2011/021982, which is incorporated herein by reference in its entirety. The synthesis of compounds of Formula II is provided in PCT/US2011/060791, which is incorporated herein by reference in its entirety. The synthesis of the compounds of Formula III is provided in U.S. Pat. Nos. 5,635,517; 6,281,230; 6,335,349; and 6,476,052; and in International Patent Application No. PCT/US97/013375, each of which is incorporated herein by reference in its entirety.

### Example 1: Synthesis of 2-(diphenylamino)-N-(7-(hydroxyamino)-7-oxoheptyl) pyrimidine-5-carboxamide (Compound A)

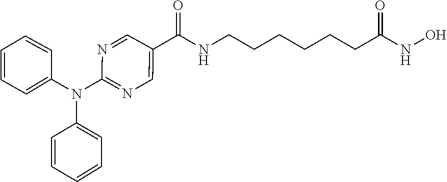 [SMILES: O=C(CCCCCCNC(=O)c1cnc(N(c2ccccc2)c2ccccc2)nc1)NO]

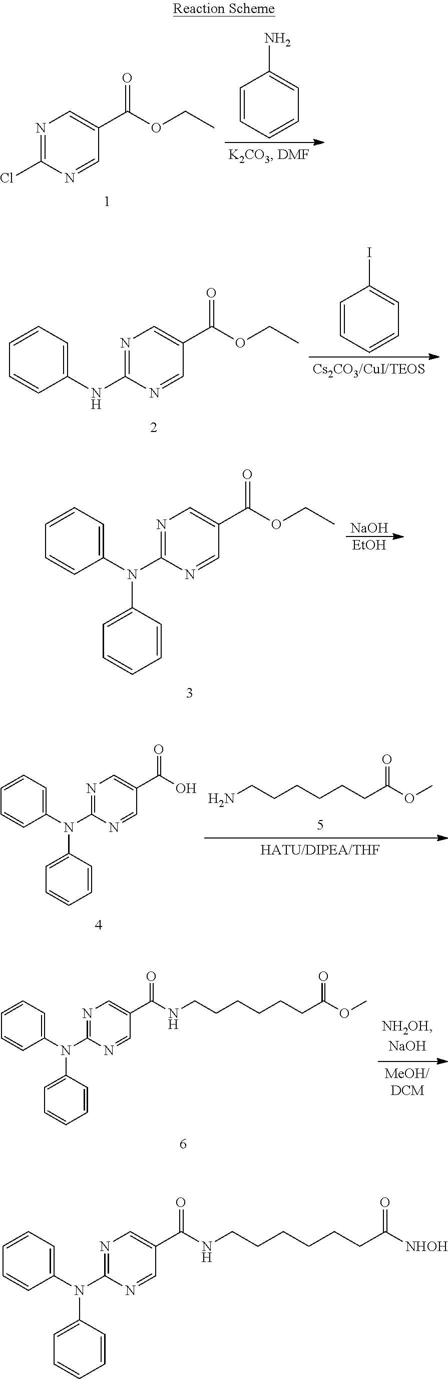

### Synthesis of Intermediate 2

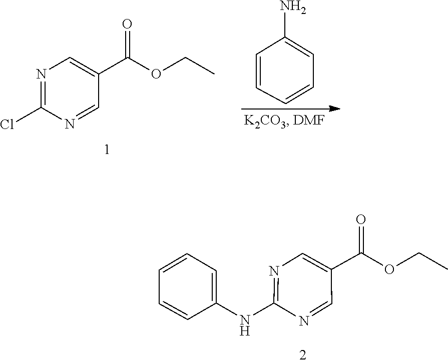

A mixture of aniline (3.7 g, 40 mmol), ethyl 2-chloropyrimidine-5-carboxylate 1 (7.5 g, 40 mmol), K₂CO₃ (11 g, 80 mmol) in DMF (100 ml) was degassed and stirred at 120° C. under N₂ overnight. The reaction mixture was cooled to rt and diluted with EtOAc (200 ml), then washed with saturated brine (200 ml×3). The organic layer was separated and dried over Na₂SO₄, evaporated to dryness and purified by silica gel chromatography (petroleum ethers/EtOAc=10/1) to give the desired product as a white solid (6.2 g, 64%).

### Synthesis of Intermediate 3

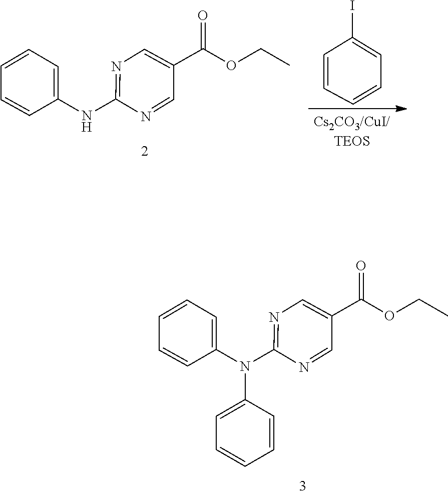

A mixture of the compound 2 (6.2 g, 25 mmol), iodobenzene (6.12 g, 30 mmol), CuI (955 mg, 5.0 mmol), Cs₂CO₃ (16.3 g, 50 mmol) in TEOS (200 ml) was degassed and purged with nitrogen. The resulting mixture was stirred at 140° C. for 14 h. After cooling to rt, the residue was diluted with EtOAc (200 ml) and 95% EtOH (200 ml), NH₄F-H₂O on silica gel [50 g, pre-prepared by the addition of NH₄F (100 g) in water (1500 ml) to silica gel (500 g, 100-200 mesh)] was added, and the resulting mixture was kept at rt for 2 h, the solidified materials was filtered and washed with EtOAc. The filtrate was evaporated to dryness and the residue was purified by silica gel chromatography (petroleum ethers/EtOAc=10/1) to give a yellow solid (3 g, 38%).

### Synthesis of Intermediate 4

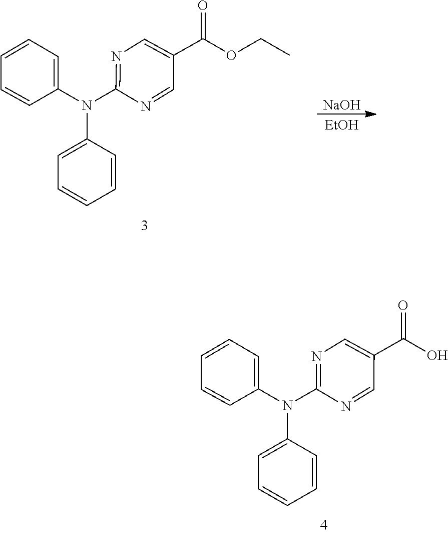 [SMILES: "EtOH" ; CCOC(=O)c1cnc(N(c2ccccc2)c2ccccc2)nc1 ; "NaOH" ; O=C(O)c1cnc(N(c2ccccc2)c2ccccc2)nc1]

2N NaOH (200 ml) was added to a solution of the compound 3 (3.0 g, 9.4 mmol) in EtOH (200 ml). The mixture was stirred at 60° C. for 30 min. After evaporation of the solvent, the solution was neutralized with 2N HCl to give a white precipitate. The suspension was extracted with EtOAc (2×200 ml), and the organic layer was separated, washed with water (2×100 ml), brine (2×100 ml), and dried over Na₂SO₄. Removal of solvent gave a brown solid (2.5 g, 92%).

### Synthesis of Intermediate 6

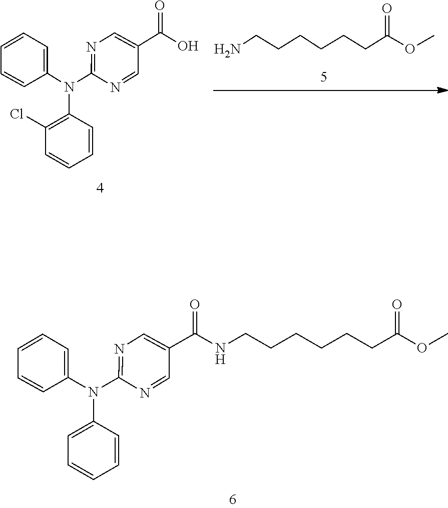 [SMILES: O=C(O)c1cnc(N(c2ccccc2)c2ccccc2Cl)nc1 ; COC(=O)CCCCCCN ; COC(=O)CCCCCCNC(=O)c1cnc(N(c2ccccc2)c2ccccc2)nc1]

A mixture of compound 4 (2.5 g, 8.58 mmol), aminoheptanoate 5 (2.52 g, 12.87 mmol), HATU (3.91 g, 10.30 mmol), DIPEA (4.43 g, 34.32 mmol) was stirred at rt overnight. After the reaction mixture was filtered, the filtrate was evaporated to dryness and the residue was purified by silica gel chromatography (petroleum ethers/EtOAc=2/1) to give a brown solid (2 g, 54%).

### Synthesis of 2-(diphenyl amino)-N-(7-(hydroxyamino)-7-oxoheptyl)pyrimidine-5 -carboxamide

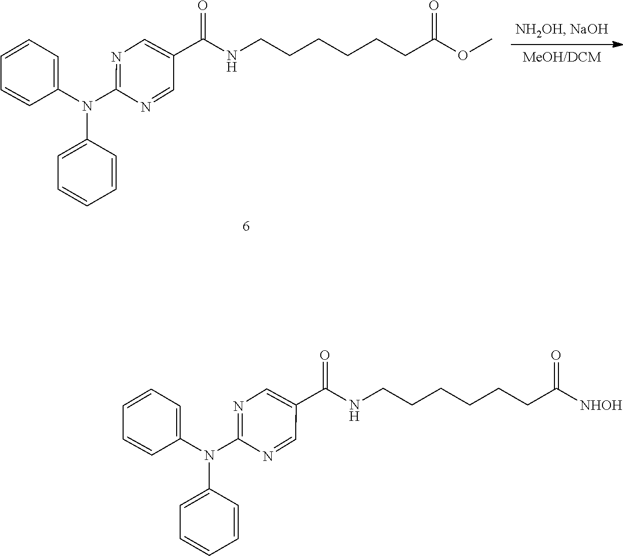

A mixture of the compound 6 (2.0 g, 4.6 mmol), sodium hydroxide (2N, 20 mL) in MeOH (50 ml) and DCM (25 ml) was stirred at 0° C. for 10 min. Hydroxylamine (50%) (10 ml) was cooled to 0° C. and added to the mixture. The resulting mixture was stirred at rt for 20 min. After removal of the solvent, the mixture was neutralized with 1M HCl to give a white precipitate. The crude product was filtered and purified by pre-HPLC to give a white solid (950 mg, 48%).

### Example 2: Synthesis of 2-((2-chlorophenyl)(phenyl)amino)-N-(7-(hydroxyamino)-7-oxoheptyl)pyrimidine-5-carboxamide (Compound B)

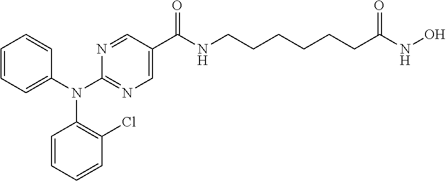 [SMILES: O=C(CCCCCCNC(=O)c1cnc(N(c2ccccc2)c2ccccc2Cl)nc1)NO]

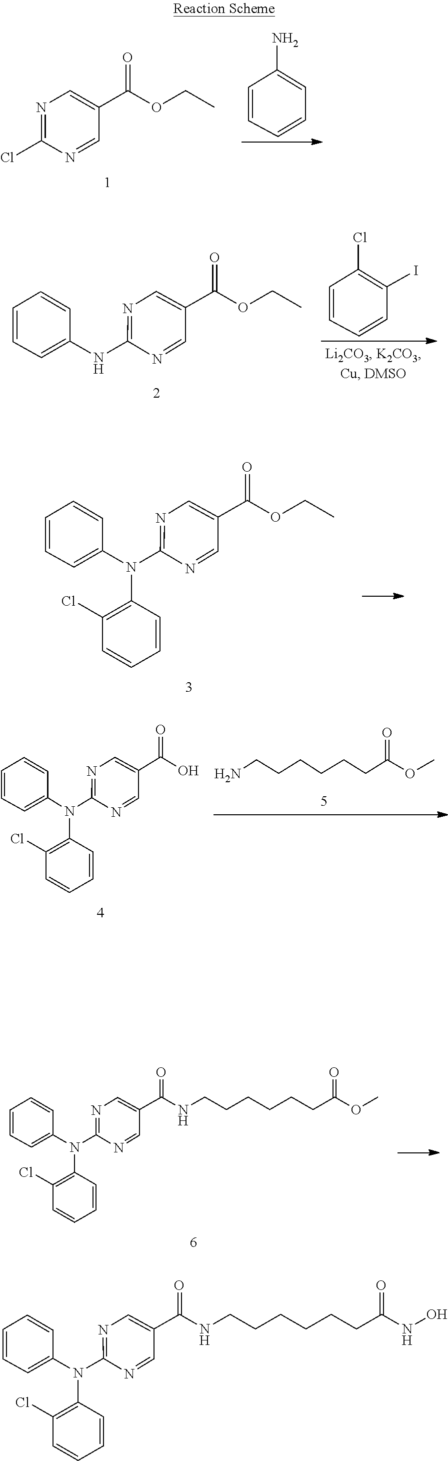

Synthesis of Intermediate 2: See synthesis of intermediate 2 in Example 1.

Synthesis of Intermediate 3: A mixture of compound 2 (69.2 g, 1 equiv.), 1-chloro-2-iodobenzene (135.7 g, 2 equiv.), Li₂CO₃ (42.04 g, 2 equiv.), K₂CO₃ (39.32 g, 1 equiv.), Cu (1 equiv. 45 μm) in DMSO (690 ml) was degassed and purged with nitrogen. The resulting mixture was stirred at 140° C. Work-up of the reaction gave compound 3 at 93% yield.

Synthesis of Intermediate 4: See synthesis of intermediate 4 in Example 1.

Synthesis of Intermediate 6: See synthesis of intermediate 6 in Example 1.

Synthesis of 2-((2-chlorophenyl)(phenyl)amino)-N-(7-(hydroxyamino)-7-oxoheptyl)pyrimidine-5-carboxamide (Compound B): See synthesis of Compound A in Example 1.

### Example 3: Synthesis of 2-((1-(3-fluorophenyl)cyclohexyl)amino)-N-hydroxypyrimidine-5-carboxamide (Compound C)

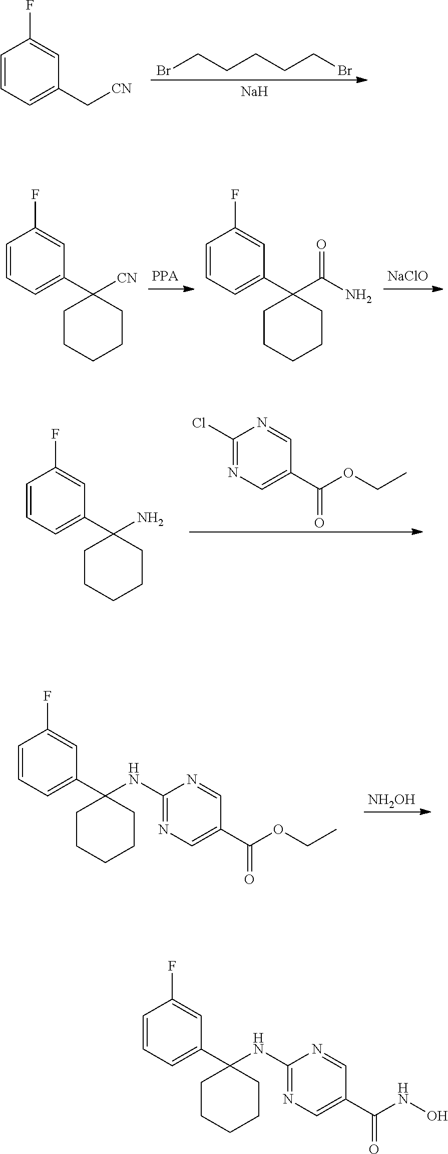

### Synthesis of 1-(3-fluorophenyl)cyclohexanecarbonitrile

To a solution of 2-(3-fluorophenyl)acetonitrile (100 g, 0.74 mol) in Dry DMF (1000 ml) was added 1,5-dibromopentane (170 g, 0.74 mol), NaH (65 g, 2.2 eq) was added dropwise at ice bath. After addition, the resulting mixture was vigorously stirred overnight at 50° C. The suspension was quenched by ice water carefully, extracted with ethyl acetate (3*500 ml). The combined organic solution was concentrate to afford the crude which was purified on flash column to give 1-(3-fluorophenyl)cyclohexanecarbonitrile as pale solid (100 g, 67%).

### Synthesis of 1-(3-fluorophenyl)cyclohexanecarboxamide

To a solution of 1-(3-fluorophenyl)cyclohexanecarbonitrile (100 g, 0.49 mol) in PPA (500 ml) was heated at 110° C. for about 5-6 hours. After completed, the resulting mixture was carefully basified with sat.NaHCO3 soultion until the PH=8-9. The precipitate was collected and washed with water (1000 ml) to afford 1-(3-fluorophenyl)cyclohexanecarboxamide as white solid (95 g, 87%).

### Synthesis of 1-(3-fluorophenyl)cyclohexanamine

To a solution of 1-(3-fluorophenyl)cyclohexanecarboxamide (95 g, 0.43 mol) in n-BuOH (800 ml) was added NaClO (260 ml, 1.4 eq), then 3N NaOH (400 ml, 2.8 eq) was added at 0° C. and the reaction was stirred overnight at r.t. The resulting mixture was extracted with EA (2*500 ml), the combined organic solution was washed with brine, dried to afford the crude which was further purification on treating with HCl salt as white powder (72 g, 73%).

### Synthesis of ethyl 2-(1-(3-fluorophenyl)cyclohexylamino)pyrimidine-5-carboxylate

To a solution of 1-(3-fluorophenyl)cyclohexanamine hydrochloride (2.29 g 10 mmol) in Dioxane (50 ml) was added ethyl 2-chloropyrimidine-5-carboxylate (1.87 g, 1.0 eq) and DIPEA (2.58 g, 2.0 eq). The mixture was heated overnight at 110-120° C. The resulting mixture was directly purified on silica gel column to afford the coupled product as white solid (1.37 g, 40%)

### Synthesis of 2-((1-(3 -fluorophenyl)cyclohexyl)amino)-N-hydroxypyrimidine-5-carboxamide

To a solution of ethyl 2-(1-(3-fluorophenyl)cyclohexylamino)pyrimidine-5-carboxylate (100 mg, 0.29 mmol) in MeOH/DCM(10 ml, 1:1) was added 50% NH₂OH in water (2 ml, excess), then sat. NaOH in MeOH (2 ml, excess) was added at 0° C. and the reaction was stirred for 3-4 hours. After completed, the resulting mixture was concentrated and acidified with 2N HCl to the PH=4-5. The precipitate was collected and washed by water (10 ml) to remove the NH₂OH and dried to afford 2-((1-(3-fluorophenyl)cyclohexyl)amino)-N-hydroxypyrimidine-5-carboxamide as white powder (70 mg, 73%).

### Example 4: Synthesis of N-hydroxy-2-((1-phenylcyclopropyl)amino)pyrimidine-5-carboxamide (Compound D)

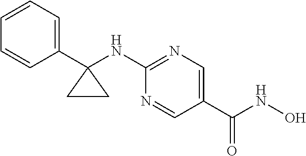 [SMILES: O=C(NO)c1cnc(NC2(c3ccccc3)CC2)nc1]

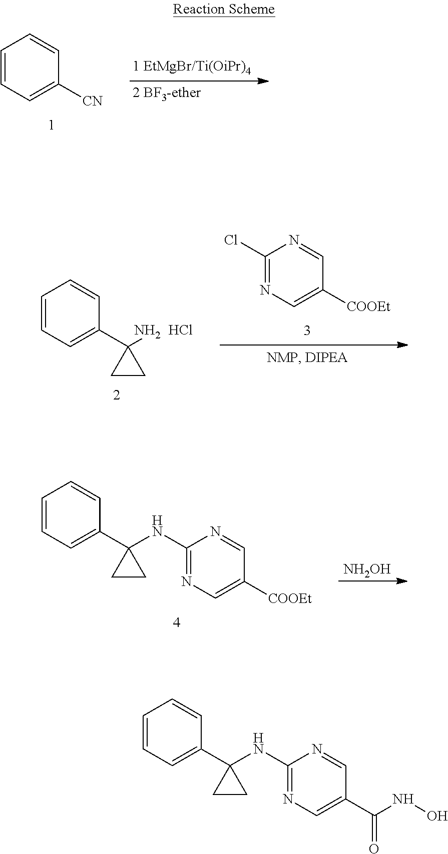

Synthesis of Intermediate 2: A solution of compound 1, benzonitrile, (250 g, 1.0 equiv.), and Ti(OiPr)₄ (1330 ml, 1.5 equiv.) in MBTE (3750 ml) was cooled to about −10 to —5 ° C. under a nitrogen atmosphere. EtMgBr (1610 ml, 3.0M, 2.3 equiv.) was added dropwise over a period of 60 min., during which the inner temperature of the reaction was kept below 5° C. The reaction mixture was allowed to warm to 15-20° C. for 1 hr. BF₃-ether (1300 ml, 2.0 equiv.) was added dropwise over a period of 60 min., while the inner temperature was maintained below 15° C. The reaction mixture was stirred at 15-20° C. for 1-2 hr. and stopped when a low level of benzonitrile remained. 1N HCl (2500 ml) was added dropwise while maintaining the inner temperature below 30° C. NaOH (20%, 3000 ml) was added dropwise to bring the pH to about 9.0, while still maintaining a temperature below 30° C. The reaction mixture was extracted with MTBE (3 L×2) and EtOAc (3 L×2), and the combined organic layers were dried with anhydrous Na₂SO₄ and concentrated under reduced pressure (below 45° C.) to yield a red oil. MTBE (2500 ml) was added to the oil to give a clear solution, and upon bubbling with dry HCl gas, a solid precipitated. This solid was filtered and dried in vacuum yielding 143 g of compound 2.

Synthesis of Intermediate 4: Compound 2 (620 g, 1.0 equiv) and DIPEA (1080 g, 2.2 equiv. were dissolved in NMP (3100 ml) and stirred for 20 min. Compound 3 (680 g, 1.02 equiv.) was added and the reaction mixture was heated to about 85-95° C. for 4 hrs. The solution was allowed to slowly cool to r.t. This solution was poured onto H₂O (20 L) and much of the solid was precipitated out from the solution with strong stirring. The mixture was filtered and the cake was dried under reduced pressure at 50° C. for 24 hr., yielding 896 g of compound 4 (solid, 86.8%).

### Synthesis of N-hydroxy-2-((1-phenylcyclopropyl)amino)pyrimidine-5-carboxamide

(Compound D): A solution of MeOH(1000 ml) was cooled to about 0-5° C. with stirring. NH₂OH HCl (1107 g, 10 equiv.) was added, followed by careful addition of NaOCH₃ (1000 g, 12.0 equiv.) The resulting mixture was stirred at 0-5° C. for one hr, and was filtered to remove the solid. Compound 4 (450 g, 1.0 equiv.) was added to the reaction mixture in one portion, and stirred at 10° C. for two hours until compound 4 was consumed. The reaction mixture was adjusted to a pH of about 8.5-9 through addition of HCl (6N), resulting in precipitation. The mixture was concentrated under reduced pressure. Water (3000 ml) was added to the residue with intense stirring and the precipitate was collected by filtration. The product was dried in an oven at 45° C. overnight (340 g, 79% yield).

### Example 5: HDAC Enzyme Assays

Compounds for testing were diluted in DMSO to 50 fold the final concentration and a ten point three fold dilution series was made. The compounds were diluted in assay buffer (50 mM HEPES, pH 7.4, 100 mM KCl, 0.001% Tween-20, 0.05% BSA, 20 μM TCEP) to 6 fold their final concentration. The HDAC enzymes (purchased from BPS Biosciences) were diluted to 1.5 fold their final concentration in assay buffer. The tripeptide substrate and trypsin at 0.05 μM final concentration were diluted in assay buffer at 6 fold their final concentration. The final enzyme concentrations used in these assays were 3.3 ng/ml (HDAC1), 0.2 ng/ml (HDAC2), 0.08 ng/ml (HDAC3) and 2 ng/ml (HDAC6). The final substrate concentrations used were 16 μM (HDAC1), 10 μM (HDAC2), 17 μM (HDAC3) and 14 μM (HDAC6). Five μl of compound and 20 μl of enzyme were added to wells of a black, opaque 384 well plate in duplicate. Enzyme and compound were incubated together at room temperature for 10 minutes. Five μl of substrate was added to each well, the plate was shaken for 60 seconds and placed into a Victor 2 microtiter plate reader. The development of fluorescence was monitored for 60 min and the linear rate of the reaction was calculated. The IC50 was determined using Graph Pad Prism by a four parameter curve fit.

### Example 6: HDAC6 Inhibitors Synergize with IMiDs in Multiple Myeloma Cell Killing

### Experiment 1

MM.1s cells were cultured for 48 hours with 0, 0.6, 1.25, or 2.5 μM lenalidomide (Compound E) or 0, 0.6, 1.25, or 2.5 μM pomalidomide (Compound F), with 0, 1, 2, or 4 μM Compound A. Cell growth was assessed by MTT assay. The Combination Index (CI) was calculated using CompuSyn software.

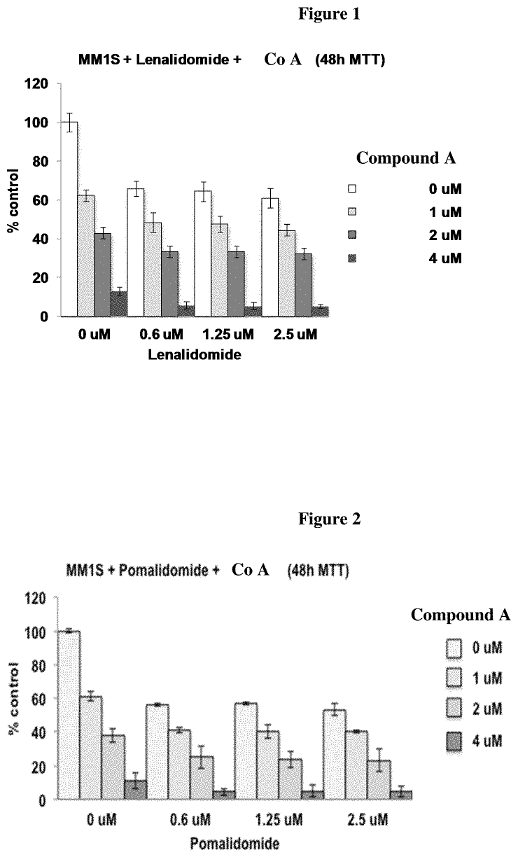

The data show that when Compound A was combined with either Compound E (lenalidomide) (see FIG. 1) or Compound F (pomalidomide) (see FIG. 2), it resulted in synergistic cytotoxicity in multiple myeloma cells in vitro. This synergy was observed within the effective clinical concentrations of both IMiDs.

### Experiment 2

These above results from Experiment 1 were further confirmed by using a highly selective HDAC6 inhibitor, Compound C, in the same experiment. Data not shown.

### Experiment 3

MM.1s cells were cultured for 48 hours with 0, 1.25, or 2.5 μM lenalidomide (Compound E) and 0, 1, 2, or 4 μM Compound A, with (50 nM) or without (0 nM) dexamethasone. Cell growth was assessed by MTT assay. The Combination Index (CI) was calculated using CompuSyn software.

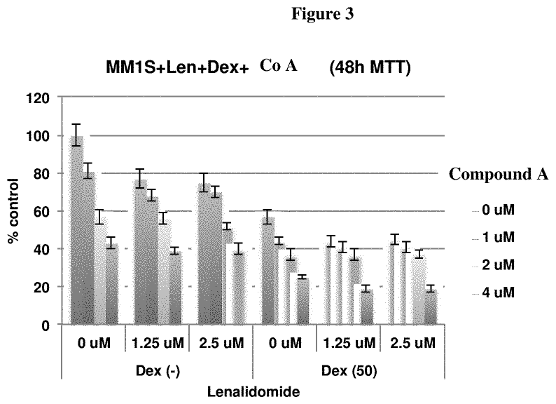

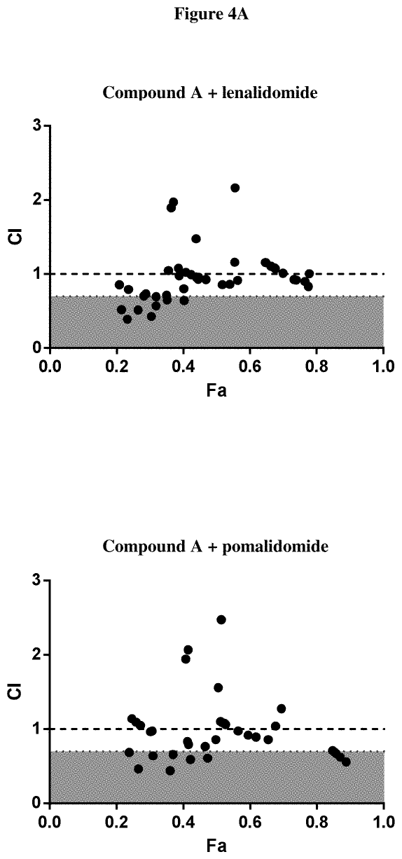

The data show that when Compound A was combined with Compound E (lenalidomide) (see FIG. 3), it resulted in synergistic cytotoxicity in multiple myeloma cells in vitro. FIG. 3 also shows that the activity observed with Compound A and Compound E is further enhanced by the addition of dexamethasone.

### Experiment 4

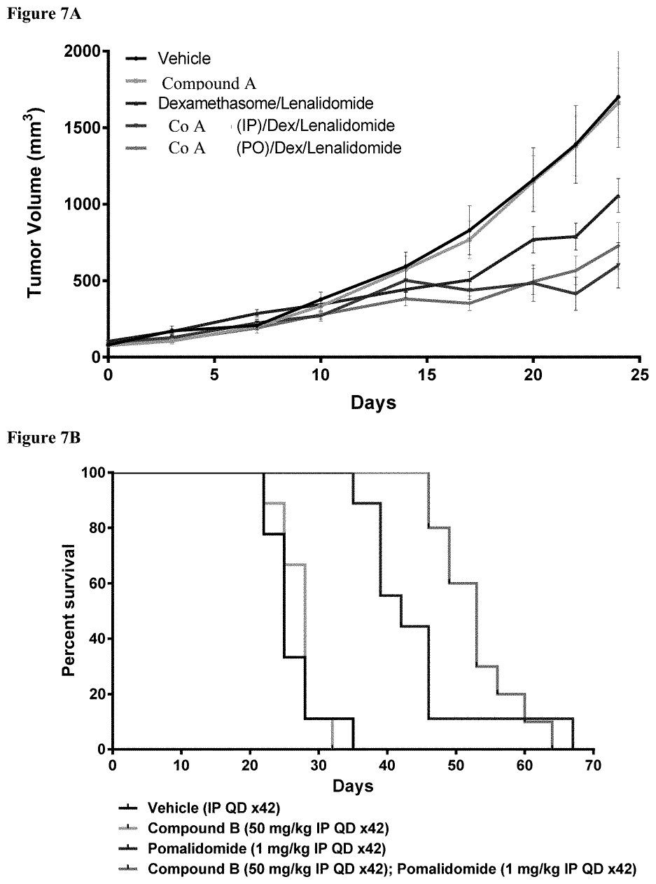

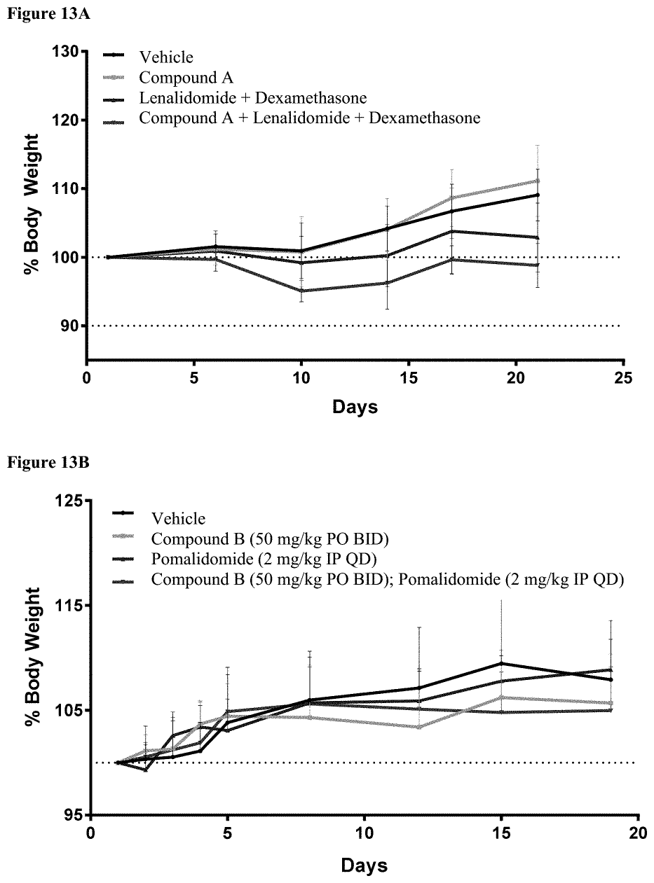

In this experiment, it is shown that combining an HDAC6 inhibitor (Compound A or Compound B) with either lenalidomide or pomalidomide leads to synergistic decreases in the viability of two different multiple myeloma cell lines in vitro (MM.1s and H929). The relevance of inhibition of HDAC6 to this synergistic effect was validated by demonstrating synergistic interactions of either IMiD molecule with Compound C, which is more than 300-fold selective for HDAC6 over class I HDAC's. Additionally, staining of H929 cells for markers of apoptosis demonstrated that treatment with a combination of Compound A plus an IMiD led to an approximately 1.6-2 fold increase in cells entering apoptosis relative to cells treated with either agent alone. Further, the combination of Compound A, lenalidomide, and dexamethasone was well tolerated in vivo with no overt evidence of toxicity (FIG. 13A), and an in vivo efficacy study with this combination in a xenograft model of multiple myeloma showed enhanced tumor growth inhibition by the triple combination over lenalidomide plus dexamethasone alone (FIG. 7A).

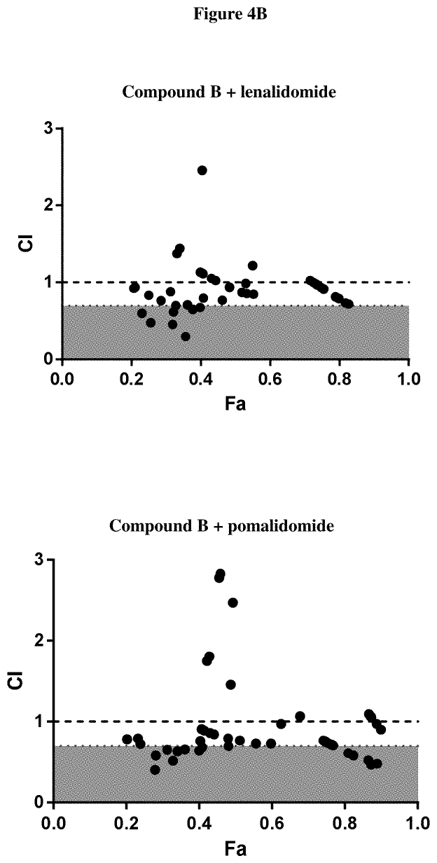

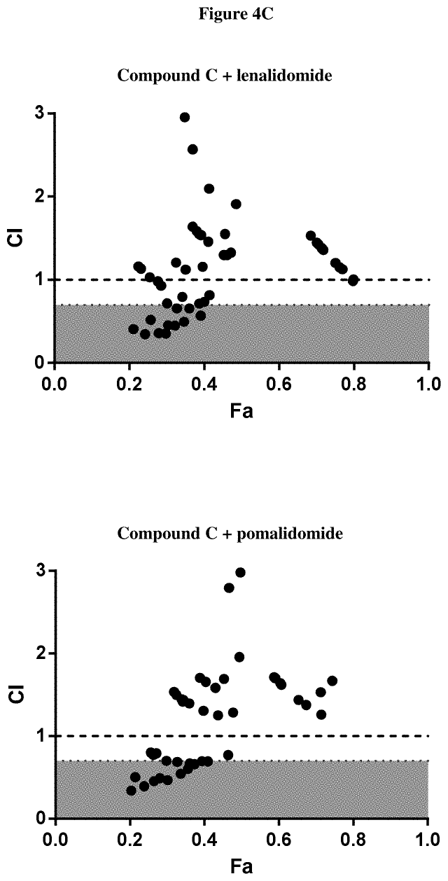

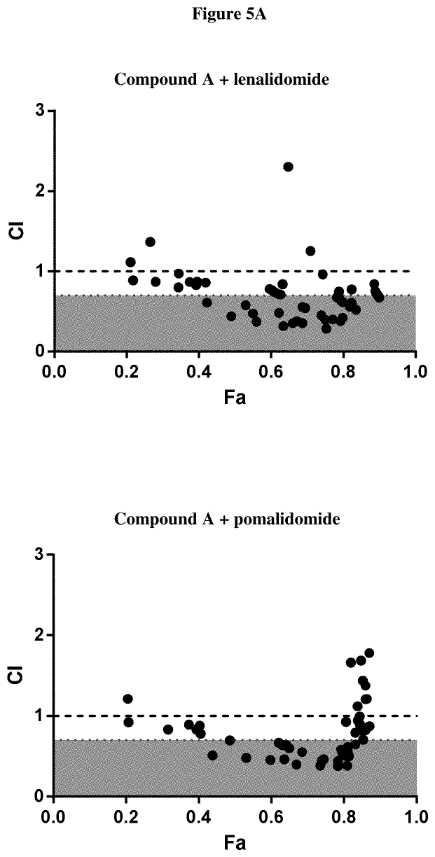

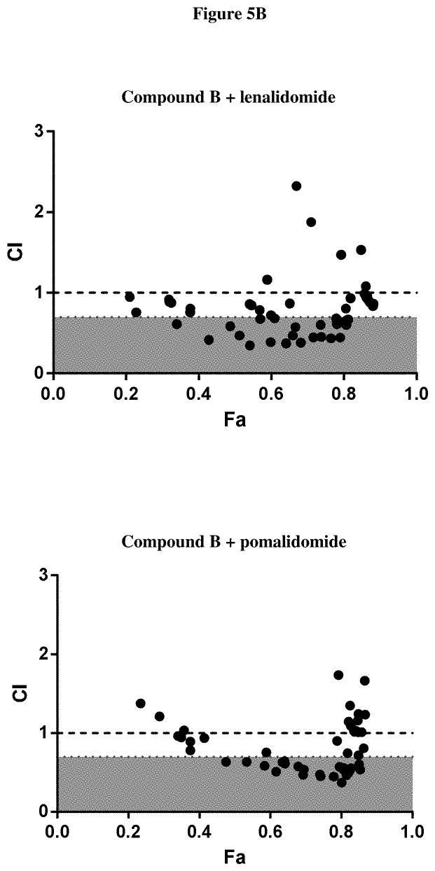

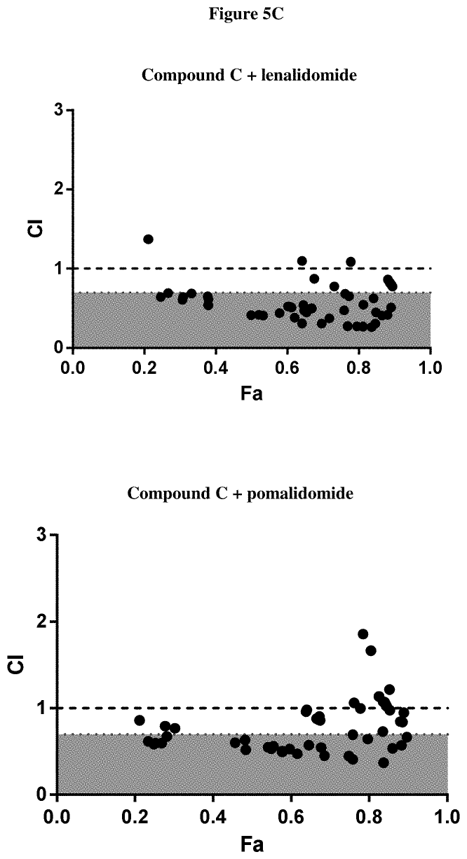

Briefly, for viability assays, cells were seeded in 384-well plates and treated in quadruplicate in a dose-matrix format with an HDAC6 inhibitor (Compound A, Compound B, or Compound C) in combination with lenalidomide or pomalidomide. After incubating these cells for 48 hr, total cell viability was assessed via an MTS assay (Aqueous One, Promega). The fraction affected (Fa) was subsequently determined for each dose combination and the combination index (CI) was assessed using the method of Chou-Talalay. CI values less than one represent a synergistic effect, values equal to one suggest an additive effect, and values greater than two indicate an antagonistic effect. As can be seen in the Fa-CI plots in FIGS. 4A-C and 5A-C, in both myeloma cell lines all HDAC6 inhibitors showed strong evidence of synergy with the tested IMiDs across a broad range of Fa's. This is evidenced by the large number of data points (representing individual dose combinations) in the Fa-CI plot that fall below the highly stringent cutoff of 0.7.

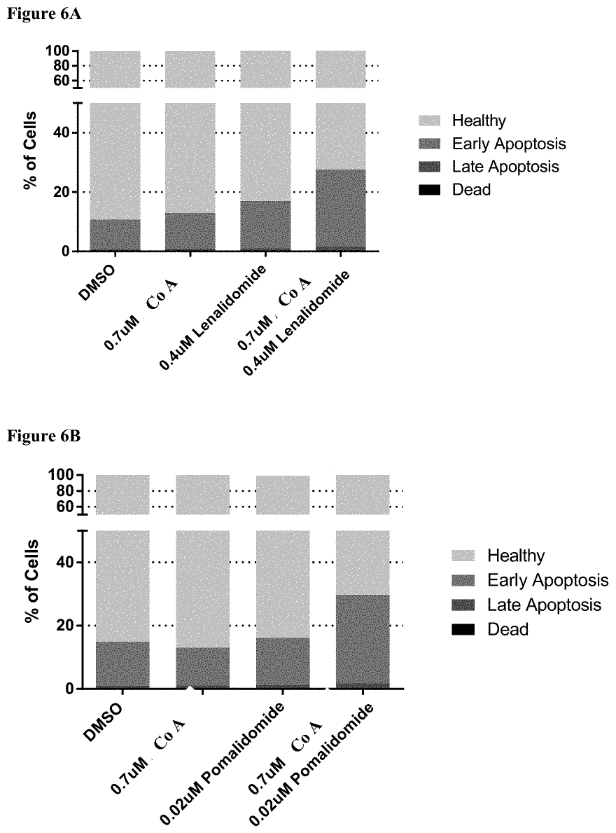

To test for the induction of apoptosis, H929 cells were treated with DMSO, 0.7 uM Compound A, 0.4 uM lenalidomide, or the combination of both drugs for 72 hours. Alternatively, H929 cells were treated for 72 hours with DMSO, 0.7 uM Compound A, 0.02 uM pomalidomide, or the combination of both drugs. Cells were then harvested and stained with Annexin V (which recognizes an epitope on cells in the early stages of apoptosis) and propidium iodide (which is excluded from cells with intact membranes, thus marking only dead cells). Flow cytometry analysis was then used to measure the number of healthy and apoptotic cells under each treatment condition. While treatment with low doses of each compound individually did not result in the induction of apoptosis, combination treatment with Compound A plus an IMiD resulted in an approximate doubling in the percentage of cells undergoing apoptosis. See FIGS. 6A-B.

For animal studies, MM.1s cells were implanted subcutaneously in immunocompromised mice. Upon establishment of tumors, the animals were separated into groups and treated with vehicle alone, Compound A alone (30 mpk IP), lenalidomide (15 mpk IP) plus dexamethasone (1 mpk IP), or lenalidomide and dexamethasone plus Compound A delivered either orally (100 mpk BID PO) or intraperitoneally (30 mpk IP). While treatment with lenalidomide plus dexamethasone delayed tumor growth in this model, the addition of Compound A to this combination resulted in even greater tumor growth inhibition. Together, these results (see FIG. 7A) provide strong evidence that inhibition of HDAC6 in combination with an IMiD results in synergistic cell killing, and further suggests that combinations of drugs targeting HDAC6 with IMiDs may provide significant clinical benefit for multiple myeloma patients.

### Example 7: HDAC6 Inhibitors with IMiDs Increase Apoptosis & Decrease c-Myc

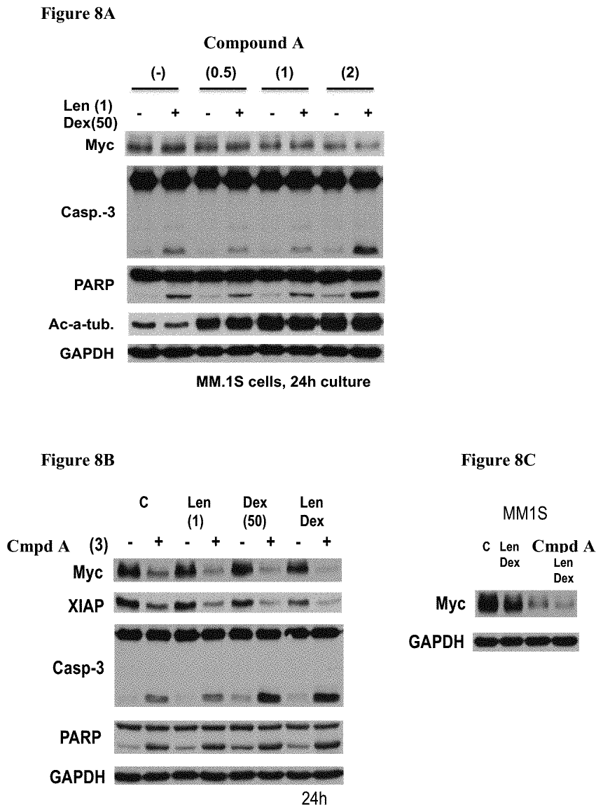

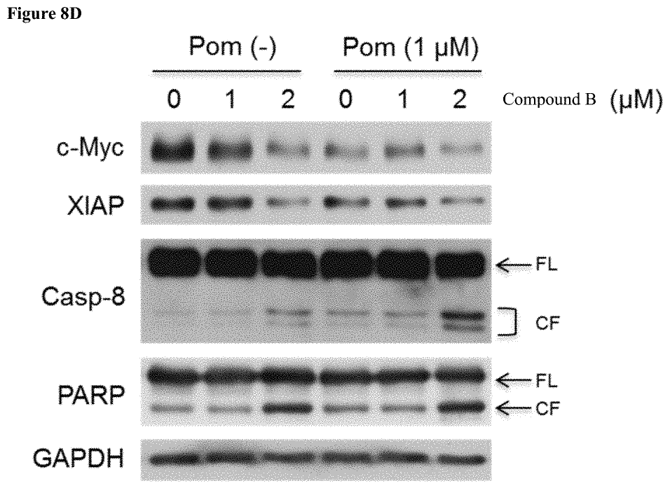

MM.1s cells were cultured for 48 hours with Compound E (1 μM) and Compound A (FIG. 8A—0.5, 1, or 2 μM; FIG. 8B—3 μM), with or without dexamethasone (50 nM). Whole cell lysates were subjected to immunoblotting using the indicated antibodies.

The data from the initial mechanistic studies showed that the induction of synergistic cytotoxicity by the combination treatment of Compound A and Compound E was due to increased apoptosis, as evidenced by caspase-3/PARP cleavage (see FIGS. 8A and 8B), which are markers of apoptosis. Previous studies have shown that c-MYC plays a crucial role in multiple myeloma pathogenesis, and that the expression of c-MYC was significantly downregulated by an immunomodulatory drug. Importantly, the downregulation of c-MYC by an immunomodulatory drug was markedly enhanced in the presence of Compound A in a dose-dependent fashion, and was associated with decreased expression of the anti-apoptotic protein XIAP (see FIGS. 8A and 8B and 8C). Thus, Compound A and Compound E with dexamethasone leads to suppression of Myc expression, a key transcipritonal regulator in cancer.

### Example 8: Compound A, a Selective HDAC6 Inhibitor, in Combination with Compound E Is Well Tolerated Without Dose Limiting Toxicity in Patients with Multiple Myeloma at Doses Demonstrating Biologic Activity: Interim Results of a Phase 1B Clinical Trial

Compound A is the first selective HDAC6 inhibitor in clinical trials and is well-tolerated as a monotherapy up to 360 mg/day, the maximum dose examined. A pharmacologically relevant Cₘₐₓ≥1 μM was achieved at dose levels >80 mg. Unlike the nonselective HDAC inhibitors, which are associated with severe fatigue, vomiting, diarrhea, and myelosuppression, dose limiting toxicities (DLTs) were not observed with Compound A. Compound A synergizes in vitro with lenalidomide (Compound E) in multiple myeloma cell lines, thus providing the rationale to conduct a Phase 1b trial of Compound A in combination with lenalidomide in patients who have progressed on at least one prior treatment regimen, who have a creatinine clearance >50 mg/mL/min, and adequate bone marrow and hepatic function. In Part A of the trial, patients were treated with escalating doses of oral Compound A in combination with a standard dose and schedule of lenalidomide and dexamethasone on days 1-5 and 8-12 of a 28 day cycle. For example, the patients in cohort 1 received 40 mg of Compound A, 15 mg of Compound E, and 40 mg of dexamethasone per day; the patients in cohort 2 received 40 mg of Compound A, 25 mg of Compound E, and 40 mg of dexamethasone per day; the patients in cohort 3 received 80 mg of Compound A, 25 mg of Compound E, and 40 mg of dexamethasone per day; the patients in cohort 4 received 160 mg of Compound A, 25 mg of Compound E, and 40 mg of dexamethasone per day; and the patients in cohort 5 received 240 mg of Compound A, 25 mg of Compound E, and 40 mg of dexamethasone per day. In Part B of the trial, the schedule includes Compound A on days 15-19 and subsequent cohorts will explore twice daily dosing as tolerated based on emerging clinical, pharmacokinetic (PK), and pharmacodynamic (PD) data. For example, the patients in cohort 6 received 160 mg of Compound A, 25 mg of Compound E, and 40 mg of dexamethasone per day; the patients in cohort 7 received 160 mg of Compound A, 25 mg of Compound E, and 40 mg of dexamethasone twice daily; and the patients in cohort 8 received 240 mg of Compound A, 25 mg of Compound E, and 40 mg of dexamethasone twice daily. Peripheral blood samples were obtained for PK and PD analysis at specified time points. PD assessment measured the fold increase of acetylated tubulin (a marker of HDAC6 inhibition) and acetylated histones (a marker of class 1 HDAC inhibition) in peripheral blood mononuclear cells (PBMC).

15 patients who progressed after 1 to >3 prior therapies were enrolled; 8 were relapsed, and 7 were relapsed-and-refractory. Patients were treated daily at up to 240 mg of Compound A. Fourteen patients had received prior lenalidomide, of which 6 were previously refractory as defined by having less than a minimal response (MR) to therapy (1) or progressive disease on either full dose or maintenance therapy (5). Patients have completed 0 to 11+ cycles of therapy with 10 patients continuing on therapy. Five patients have discontinued therapy due to progressive disease (PD) (3), travel difficulties (1), or missed doses of lenalidomide (1). The latter patient was replaced.

The most common treatment emergent events were fatigue (43%), upper respiratory infection (36%), anemia and peripheral edema (21% each), neutropenia (29%), and muscle spasms (21%). Most were grade 1 and 2, and there was no dose relationship to Compound A. There were 9 grade 3 and 4 events in 6 patients, primarily hematologic and also including fatigue and asymptomatic laboratory investigations. Only 1, neutropenia, was considered possibly related to Compound A by the investigator.

PK and PD data is available from 12 patients up to 160 mg dose level. PK for Compound A is similar to the analogous dose levels in phase 1a monotherapy suggesting coadministration of lenalidomide does not significantly impact the PK of Compound A. Maximal levels were ≥1 μM at ≥80 mg correlating with measurable increases >2× in acetylated tubulin with a minimal increase in acetylated histones.

Twelve patients, at doses up to 160 mg of Compound A, are evaluable for response (after at least two cycles). In addition, 1 patient who discontinued therapy after one cycle has response data available. Nine patients (69%) have ≥PR, including 1 CR, 4 VGPR, 3 PR, and 1 PRu. Two patients each had MR and SD as the best response. Reponses are durable up to 1130 cycles of therapy. Of the patients who were refractory to lenalidomide, there were 1 PR, 1 VGPR, 2 MR, and 2 SD.

Thus, Compound A can be combined with lenalidomide at doses that have biological activity, as determined by PD data in PBMC. Responses are observed, including in patients previously refractory to lenalidomide.

### Example 9: Combinations of HDAC6 Inhibitors and IMiDs Results in Synergistic Decreases in Myeloma Cell Growth and Viability

This example shows that the combination of HDAC6 inhibitors and IMiDs results in synergistic decreases in myeloma cell growth and viability.

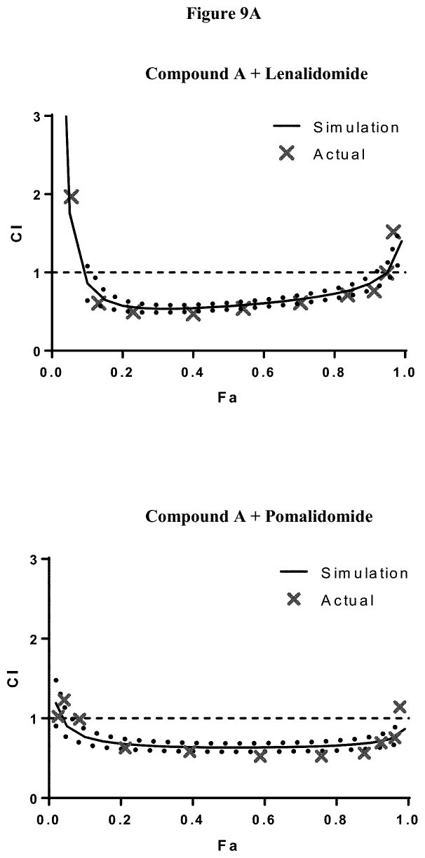

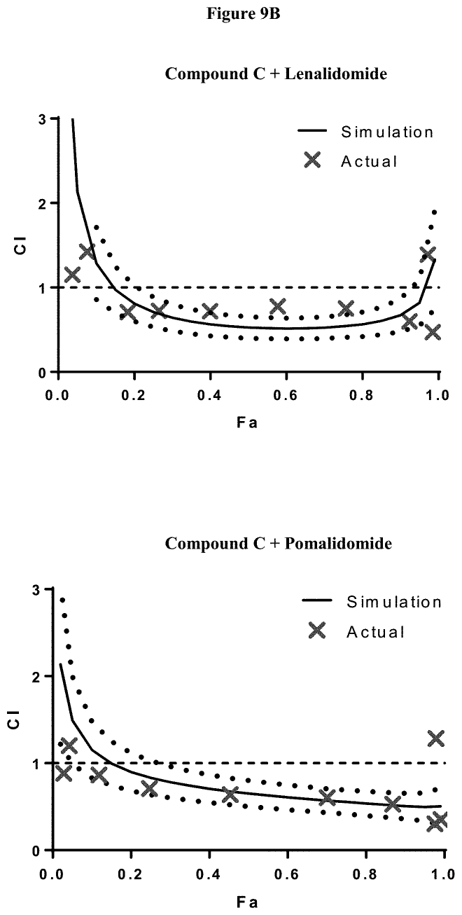

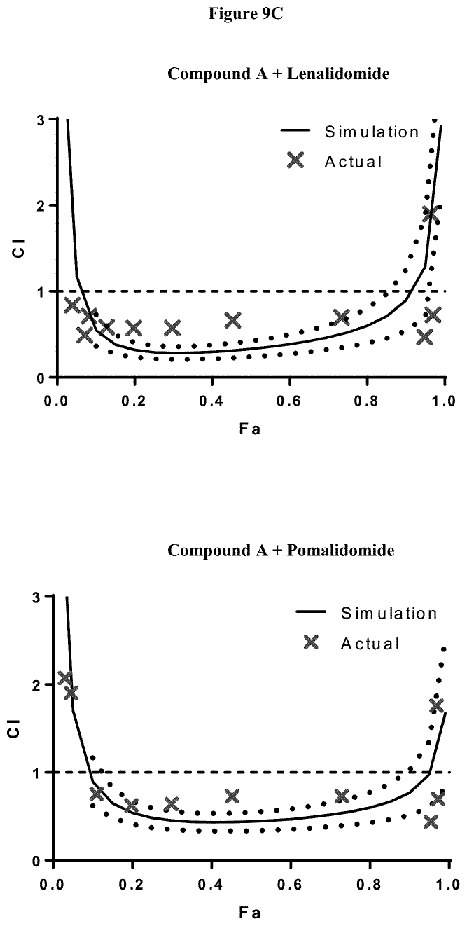

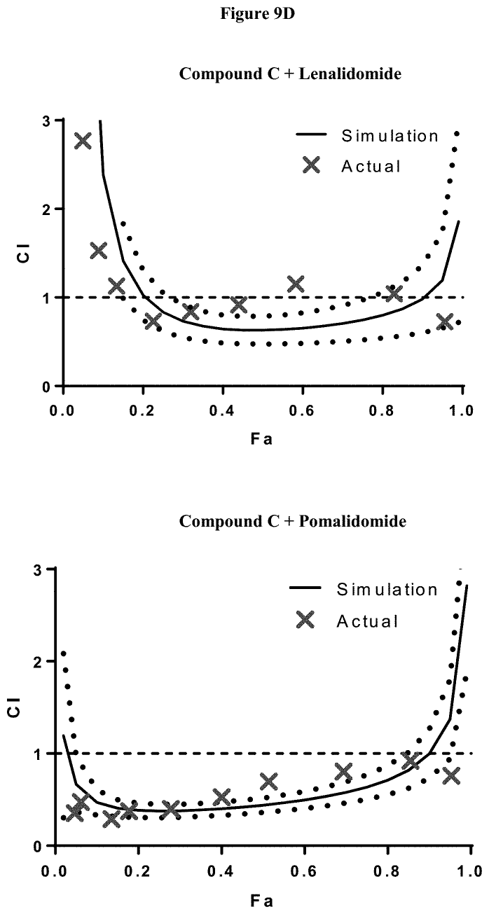

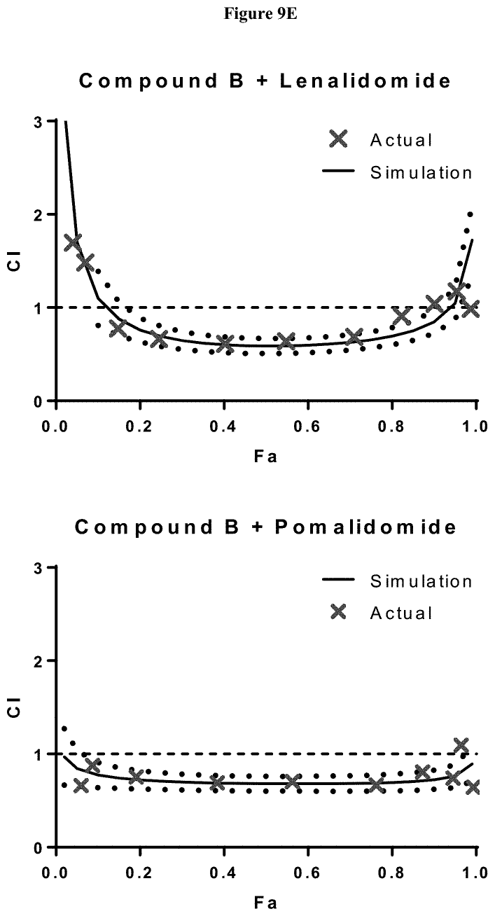

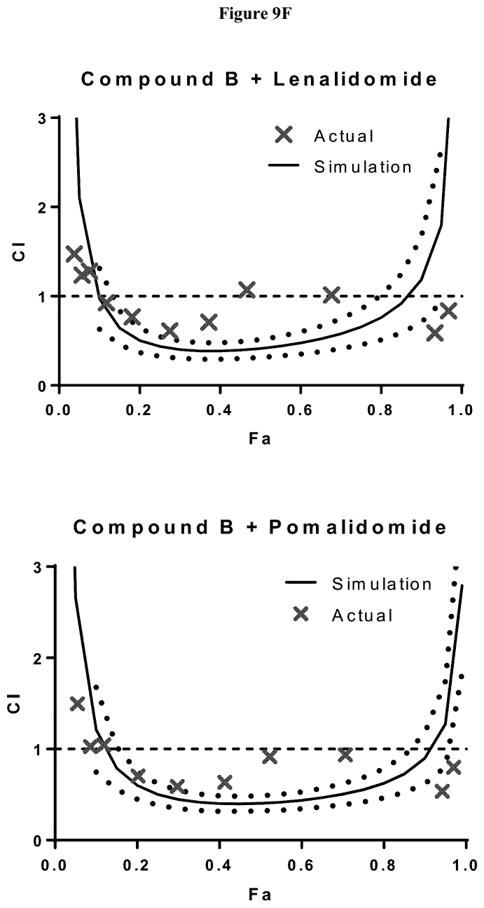

H929 (FIGS. 9A & 9B) or MM.1s (FIGS. 9C & 9D) myeloma cells were exposed to increasing doses of the HDAC6 inhibitors Compound A (FIGS. 9A & 9C) or Compound C (FIGS. 9B & 9D) alone or in combination with lenalidomide (FIGS. 9A & 9C) or pomalidomide (FIGS. 9B & 9D). A constant ratio was maintained between the dose of the HDAC6i and IMiD, and cell viability was assessed at 72 hr by MTS assay. Calcusyn software was then used to determine the combination index (CI) value at each dose combination and the relative fraction affected (F_{A}) (Actual), and a simulation was run to estimate the CI value across the entire F_{A} range (Simulation). The measurement of CI values less than 1 in all combinations strongly support a synergistic interaction between the HDAC6i and IMiDs tested.

### Example 10: The Combination of an HDAC6 Inhibitor and IMiDs Affects Cellular Proliferation and Cell Cycle Progression

This example shows that treatment of multiple myeloma cells with Compound A and/or IMiDs results in decreased cell cycle progression.

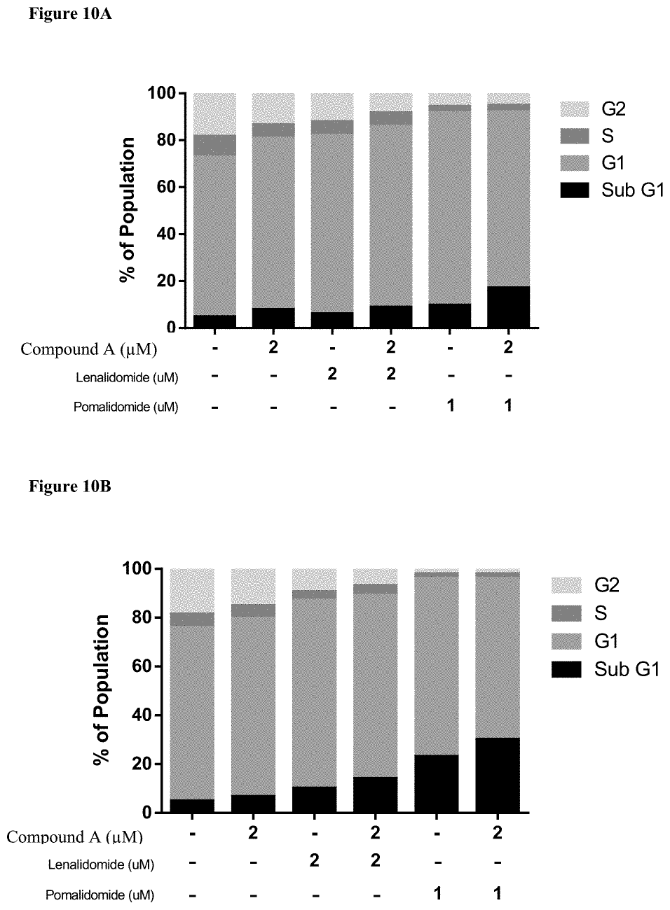

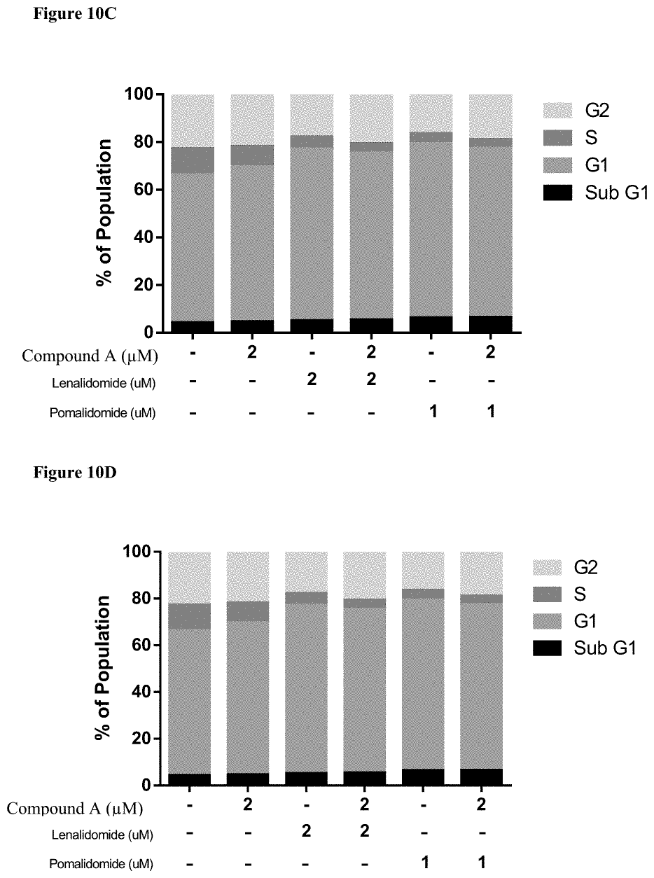

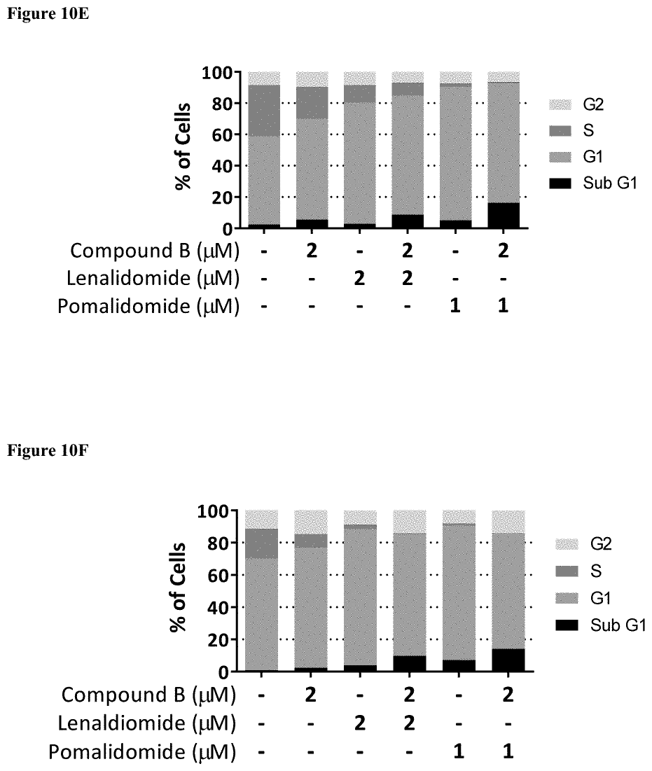

H929 (FIGS. 10A & 10B) or MM.1s (FIGS. 10C & 10D) myeloma cells were exposed to drug for 3 (FIGS. 10A & 10C) and 5 (FIGS. 10B & 10D) days and cell cycle distribution was assessed by flow cytometry via incorporation of propidium iodide. The relative fraction of cells in each stage of the cell cycle (GO/G1, S, and G2/M) as well as the fraction of dead cells (Sub G1) was then estimated. The cells were treated with DMSO, Compound A (2 μM), Lenalidomide (2 μM), Pomalidomide (1 μM), or combinations of Compound A with either IMiD. Treatment with Compound A resulted in a small reduction of cells undergoing division in S phase, while treatment with either IMiD, alone or in combination with Compound A, led to a reduction in the percentage of cells in the S and G2/M phases and a concomitant increase in cells in G0/G1. These results are consistent with decreased proliferation in response to treatment with Compound A and/or IMiDs that accumulates with prolonged exposure to the drug combination.

### Example 11: The Combination of an HDAC6 Inhibitor and IMiDs Induces Apoptosis in Multiple Myeloma Cells

This example shows that treatment of multiple myeloma cells with Compound A plus IMiDs results in synergistic increases in cellular apoptosis.

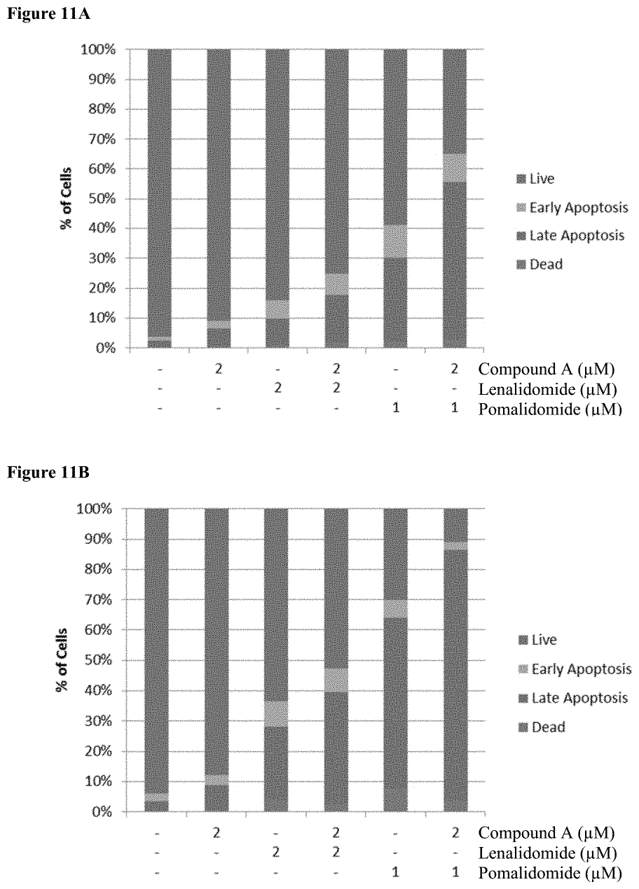

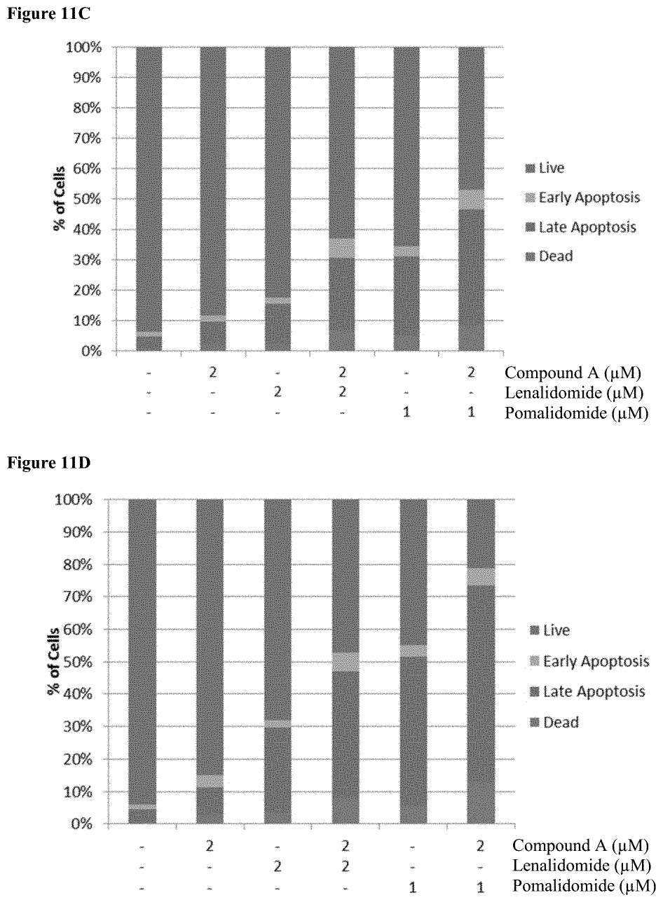

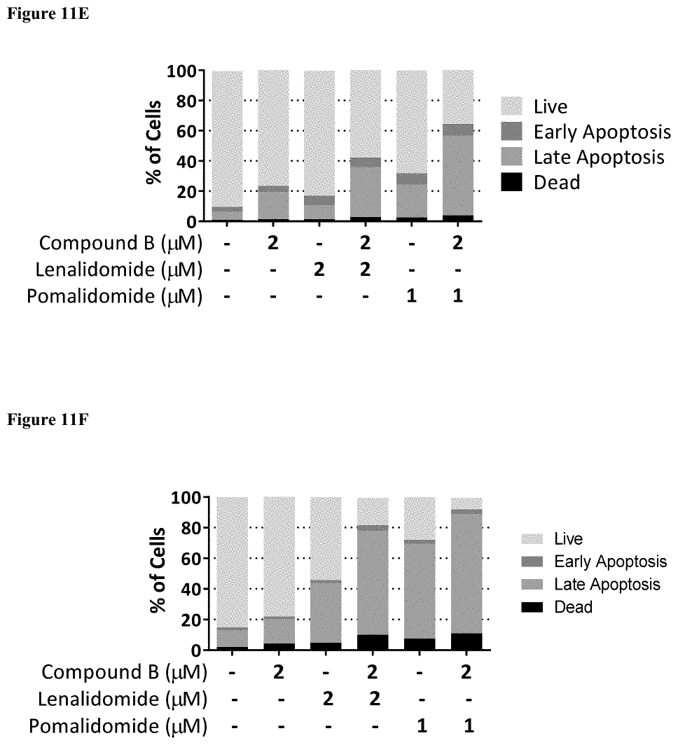

H929 (FIGS. 11A & 11B) or MM.1s (FIGS. 11C & 11D) myeloma cells were exposed to drug for 5 (FIGS. 11A & 11C) and 7 (FIGS. 11B & 11D) days, and apoptosis was assessed by flow cytometry by measuring Annexin V binding and cellular permeability to propidium iodide. The relative fraction of cells that were live, in early apoptosis, in late apoptosis, or dead was then determined. The cells were treated with DMSO, Compound A (2 μM), Lenalidomide (2 μM), Pomalidomide (1 μM), or combinations of Compound A with either IMiD. Treatment with Compound A (2 μM) resulted in a small increase in apoptosis relative to control cells, while treatment with either IMiD resulted in significantly more apoptotic cells at both time points. However, the combination of Compound A with either IMiD resulted in synergistic increases in the percentage of apoptotic cells. The percentage of cells actively undergoing apoptosis also increased with longer exposure times to the drug combinations.

### Example 12: The Combination of an HDAC6 Inhibitor and IMiDs Decreases mRNA and Protein Expression Level of MYC, IRF4, and CRBN, and Increases P21 Expression

This example shows that the expression level of MYC, IRF4, and CRBN are decreased by treatment with Compound A and IMiDs, while expression of P21 is increased by treatment with this combination.

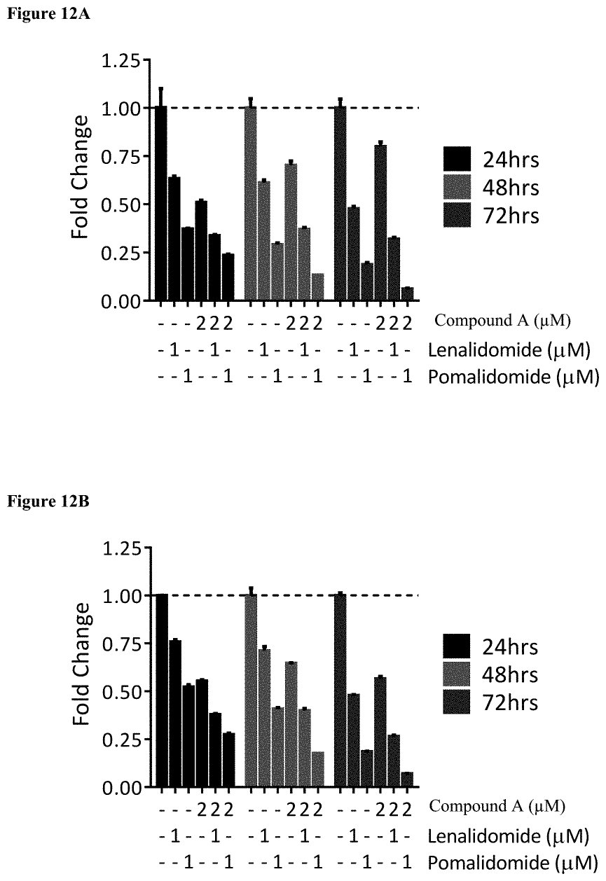

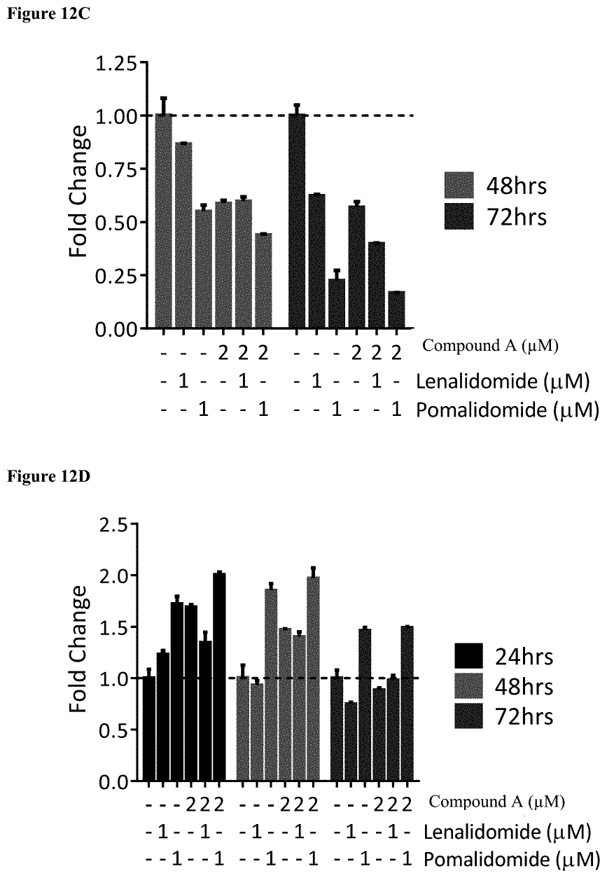

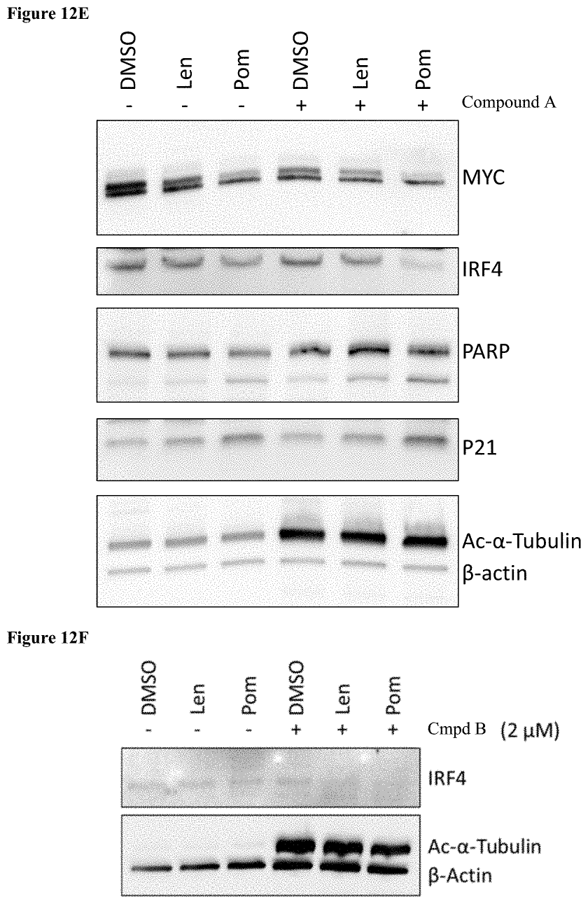

H929 myeloma cells were treated with DMSO, Compound A (2 Lenalidomide (1 μM), Pomalidomide (1 μM), or combinations of Compound A with either IMiD, and total RNA was harvested 24, 48, and 72 hours later. Quantitative reverse transcription PCR was then performed to assess the relative transcript levels of MYC (FIG. 12A), IRF4 (FIG. 12B), CRBN (FIG. 12C), and P21 (FIG. 12D) at each time point. MYC and IRF4 are critical transcription factors that are overexpressed in multiple myeloma cells, and myeloma cells were previously shown to exhibit dependence on both transcripts (Nature, 454: 226; Blood, 120: 2450), while expression of CRBN was previously shown to be inhibited by treatment of cells with IMiDs. While all three genes were decreased by all single agent treatments, combination treatment with Compound A and either IMiD resulted in further decreases in expression of these important transcripts. P21 is an inhibitor of the cell cycle, and thus increased expression of P21 would be expected to inhibit proliferation. The reduction of MYC and IRF4, and the increase of P21 expression, was confirmed at the protein level by immunoblot in H929 cells after 48 hours of combination treatment (FIG. 12E). Induction of apoptosis was also confirmed by the induction of PARP cleavage by combination treatment. Inhibition of HDAC6 by Compound A was confirmed by the detection of hyperacetylation of α-tubulin.

### Example 13: The Combination of an HDAC6 Inhibitor, lenalidomide, and dexamethasone is Well Tolerated

This example shows that the combination of an HDAC6 inhibitor, an IMiD, and dexamethasone is well tolerated in mice.

SCID-beige mice were treated with Vehicle, Compound A alone, lenalidomide plus dexamethasone, or the triple combination of lenalidomide, dexamethasone, and Compound A. Percent body weight change was determined relative to the start of dosing, and the mean change ±SD was plotted. All treatments were dosed five days per week for 3 cycles: Compound A at 100 mpk PO BID, lenalidomide at 15 mpk IP QD, and dexamethasone at 5 mpk IP QD. All treatments were well tolerated with no overt evidence of toxicity and complete recovery after minimal body weight loss. See FIG. 13A.

### Example 14: Compound B, a Selective Inhibitor of HDAC6, Synergizes with Immunomodulatory Drugs (IMiDs) in Multiple Myeloma (MM) Cells

Histone deacetylase (HDAC) enzymes represent attractive therapeutic targets in MM, but non-selective HDAC inhibitors have led to dose-limiting toxicities in patients, particularly in combination with other therapeutic agents. Ricolinostat (Compound A), a first-in-class orally available HDAC inhibitor that is 11-fold selective for HDAC6, synergizes in vitro and in vivo with bortezomib in preclinical models of MM (Blood, 20[210]: 4061), and has thus far demonstrated an improved safety and tolerability profile in Phase I trials (Raje, et al, EHA, 2014). Based on these findings, Compound B is being developed as a second generation, orally available, isoform selective inhibitor of HDAC6 for clinical evaluation in MM.

In support of the ongoing clinical development program for Compound B in MM, it is shown here that combining Compound B with either IMiD leads to synergistic decreases in the viability of MM cells in vitro. FIGS. 9E-F are sets of graphs showing that the combination of HDAC6 inhibitors and IMiDs resulted in synergistic decreases in myeloma cell growth and viability. FIG. 9E shows the results of experiments in which H929 myeloma cells were exposed to increasing doses of Compound B in combination with lenalidomide (top panel) or pomalidomide (bottom panel) at constant ratios. FIG. 9F shows the results of experiments in which MM.1s myeloma cells were exposed to increasing doses of Compound B in combination with lenalidomide (top panel) or pomalidomide (bottom panel) at constant ratios.

Time course studies demonstrated accumulation of cell cycle arrest in cells after prolonged exposure to either IMiD, as well as progressive induction of apoptosis in these cells. Notably, though, the addition of Compound B to either IMiD resulted in synergistic increases in the percentage of MM cells undergoing apoptosis. FIGS. 10E-F are graphs showing that treatment of multiple myeloma cells with Compound B and/or IMiDs resulted in decreased cell cycle progression. FIG. 10E shows the effect of treatment of H929 myeloma cells for 4 days with DMSO, Compound B (2 μM), Lenalidomide (2 μM), Pomalidomide (1 μM), or combinations of Compound B with either IMiD on cell cycle inhibition. FIG. 10F shows the effect of treatment of MM1s myeloma cells for 5 days with DMSO, Compound B (2 μM), Lenalidomide (2 μM), Pomalidomide (1 μM), or combinations of Compound B with either IMiD on cell cycle inhibition. FIGS. 11E-F are graphs showing that treatment of multiple myeloma cells with Compound B and IMiDs resulted in synergistic increases in cellular apoptosis. FIG. 11E shows the effect of treatment of H929 myeloma cells for 4 days with DMSO, Compound B (2 μM), Lenalidomide (2 μM), Pomalidomide (1 μM), or combinations of Compound B with either IMiD on the induction of apoptosis. FIG. 11F shows the effect of treatment of MM1s myeloma cells for 5 days with DMSO, Compound B (2 μM), Lenalidomide (2 μM), Pomalidomide (1 μM), or combinations of Compound B with either IMiD on the induction of apoptosis.

At the molecular level, MM cells are known to be dependent on expression of the MYC and IRF4 transcription factors. FIG. 8D shows an image of an immunoblot from MM1s cells showing that the combination of Compound B and pomalidomide (Compound F) led to suppression of Myc expression, a key transcriptional regulator in cancer. Markers of apoptosis (cleaved PARP and caspase) were increased, and suppressors of apoptosis, such as XIAP, were decreased by combination treatment. FIG. 12F is an image of an immunoblot confirming, at the protein level in H929 cells, the reduction of IRF4 after 48 hours of combination treatment with Compound B and either lenalidomide or pomalidomide relative to any of the single agents. Thus, treatment with IMiDs reduced expression of the critical genes MYC and IRF4, which were reduced even further by treatment with Compound B plus either IMiD. The molecular mechanism underlying this effect is currently being explored, though retention of low level inhibition of HDAC1, 2, and 3 by Compound B may contribute to the enhanced effects on gene expression reported here in combination with IMiDs.

Mice carrying H929 tumor xenografts were treated with DMSO, Compound B (50 mg/kg IP QD), pomalidomide (1 mg/kg IP QD), or the combination of Compound B (50 mg/kg IP QD) and pomalidomide (1 mg/kg IP QD) daily for up to 42 days. The combination showed increased overall survival relative to either single agent. See FIG. 7B. FIG. 13B is a graph showing the effects of treatment with Vehicle, Compound B alone, pomalidomide alone, or the combination of pomalidomide and Compound B on the body weight of CB17-SCID mice. These treatments were very well tolerated with no weight loss and no evidence of overt toxicity.

By demonstrating a similar tolerability and efficacy profile to ricolinostat (Compound A), these findings provide support for the clinical evaluation of Compound B in combination with IMiDs in MM patients.

### Incorporation by Reference

The contents of all references (including literature references, issued patents, published patent applications, and co-pending patent applications) cited throughout this application are hereby expressly incorporated herein in their entireties. Unless otherwise defined, all technical and scientific terms used herein are accorded the meaning commonly known to one with ordinary skill in the art.

### Equivalents

Those skilled in the art will recognize, or be able to ascertain using no more than routine experimentation, many equivalents of the specific embodiments of the invention described herein. Such equivalents are intended to be encompassed by the following claims.

1. A pharmaceutical combination for treating multiple myeloma comprising a therapeutically effective amount of a histone deacetylase 6 (HDAC6) specific inhibitor or a pharmaceutically acceptable salt thereof, and an immunomodulatory drug (IMiD) or a pharmaceutically acceptable salt thereof, wherein the HDAC6 inhibitor is a compound of Formula II: 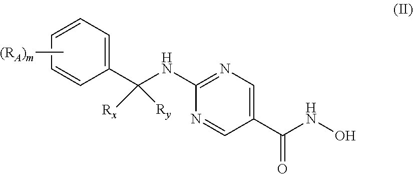
or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, cyclooctyl;
each R_{A} is independently C₁₋₆-alkyl, C₁₋₆-alkoxy, halo, OH, —NO₂, —CN, or —NH₂; and
m is 0 or 1.

2-8. (canceled)

9. A pharmaceutical combination for treating multiple myeloma comprising a therapeutically effective amount of a histone deacetylase 6 (HDAC6) specific inhibitor or a pharmaceutically acceptable salt thereof, and an immunomodulatory drug (IMiD) or a pharmaceutically acceptable salt thereof, wherein the HDAC6 specific inhibitor is a compound of Formula I: 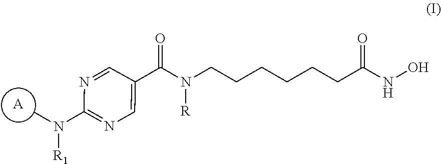
or a pharmaceutically acceptable salt thereof,
wherein,
ring B is aryl or heteroaryl;
R₁ is an aryl or heteroaryl, each of which may be optionally substituted by OH, halo, or C₁₋₆-alkyl; and
R is H or C₁₋₆-alkyl.

10. (canceled)

11. The combination of claim 9, wherein the compound of Formula I is: 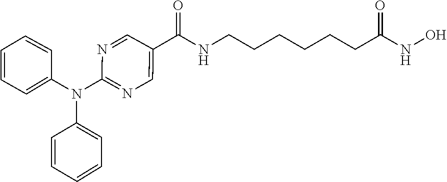 [SMILES: O=C(CCCCCCNC(=O)c1cnc(N(c2ccccc2)c2ccccc2)nc1)NO]
or a pharmaceutically acceptable salt thereof.

12. The combination of claim 9, wherein the compound of Formula I is: 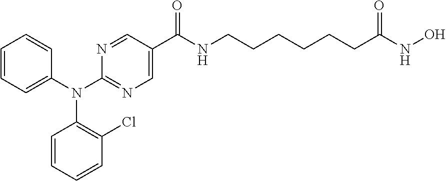 [SMILES: O=C(CCCCCCNC(=O)c1cnc(N(c2ccccc2)c2ccccc2Cl)nc1)NO]
or a pharmaceutically acceptable salt thereof.

13-15. (canceled)

16. The combination of claim 9, wherein the immunomodulatory drug is a compound of Formula III: 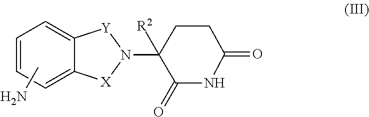
or a pharmaceutically acceptable salt thereof,
wherein,
one of X and Y is C═O, the other of X and Y is CH₂ or C═O; and
R² is H or C₁₋₆-alkyl.

17. The combination of claim 16, wherein the compound of Formula III is: 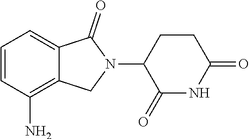 [SMILES: Nc1cccc2c1CN(C1CCC(=O)NC1=O)C2=O]
or a pharmaceutically acceptable salt thereof.

18. The combination of claim 16, wherein the compound of Formula III is: 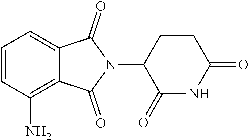 [SMILES: Nc1cccc2c1C(=O)N(C1CCC(=O)NC1=O)C2=O]
or a pharmaceutically acceptable salt thereof.

19-26. (canceled)

27. A method for treating multiple myeloma in a subject in need thereof comprising administering to the subject a therapeutically effective amount of a pharmaceutical combination comprising a histone deacetylase 6 (HDAC6) specific inhibitor or a pharmaceutically acceptable salt thereof, and an immunomodulatory drug (IMiD) or a pharmaceutically acceptable salt thereof, wherein the HDAC6 specific inhibitor is a compound of Formula I: 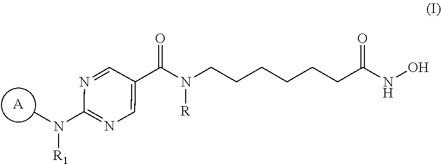
or a pharmaceutically acceptable salt thereof,
wherein,
ring B is aryl or heteroaryl;
R₁ is an aryl or heteroaryl, each of which may be optionally substituted by OH, halo, or C₁₋₆-alkyl; and
R is H or C₁₋₆-alkyl.

28. (canceled)

29. The method of claim 27, wherein the compound of Formula I is: 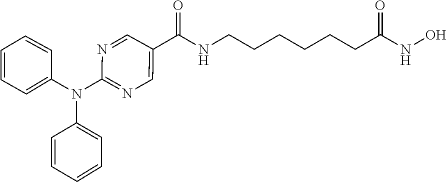 [SMILES: O=C(CCCCCCNC(=O)c1cnc(N(c2ccccc2)c2ccccc2)nc1)NO]
or a pharmaceutically acceptable salt thereof.

30. The method of claim 27, wherein the compound of Formula I is: 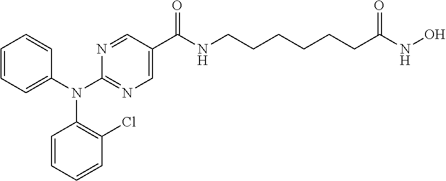 [SMILES: O=C(CCCCCCNC(=O)c1cnc(N(c2ccccc2)c2ccccc2Cl)nc1)NO]
or a pharmaceutically acceptable salt thereof.

31. (canceled)

32. (canceled)

33. (canceled)

34. The method of claim 27, wherein the immunomodulatory drug is a compound of Formula III: 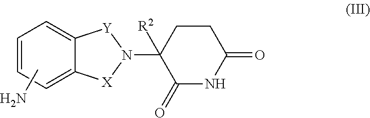
or a pharmaceutically acceptable salt thereof,
wherein,
one of X and Y is C═O, the other of X and Y is CH₂ or C═O; and
R² is H or C₁₋₆-alkyl.

35. The method of claim 34, wherein the compound of Formula III is: 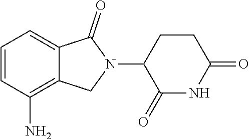 [SMILES: Nc1cccc2c1CN(C1CCC(=O)NC1=O)C2=O]
or a pharmaceutically acceptable salt thereof.

36. The method of claim 34, wherein the compound of Formula III is: 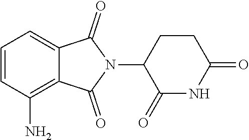 [SMILES: Nc1cccc2c1C(=O)N(C1CCC(=O)NC1=O)C2=O]
or a pharmaceutically acceptable salt thereof.

37. The method of claim 27, wherein the subject was previously refractory to an immunomodulatory drug.

38. The method of claim 27, wherein the HDAC inhibitor and the immunomodulatory drug are administered in separate dosage forms.

39. (canceled)

40. The method of claim 27, wherein the HDAC inhibitor and the immunomodulatory drug are administered at different times.

41. The method of claim 27, wherein the HDAC inhibitor and the immunomodulatory drug administered at substantially the same time.

42. (canceled)

43. (canceled)

44. (canceled)

45. (canceled)

46. (canceled)

47. The method of claim 27, wherein the combination further comprises an anti-inflammatory agent.

48. The method of claim 47, wherein the anti-inflammatory agent is dexamethasone.

49. The method of claim 27, wherein the HDAC6 specific inhibitor is administered orally.
